(19) United States
(12) Patent Application Publication
(10) Pub. No.: US 2018/0230101 A1
(43) Pub. Date: Aug. 16, 2018

(54) PROCESS FOR THE PREPARATION OF 5-AMINO-QUINOLIN-2(1H)-ONES AND THEIR TAUTOMER FORMS 5-AMINO-QUINOLIN-2-OLS

(71) Applicant: BAYER PHARMA AKTIENGESELLSCHSAFT, Berlin (DE)

(72) Inventors: Friedrich August MUEHLTHAU, Kelkheim-Fischbach (DE); Dirk BROHM, Mettmann (DE)

(21) Appl. No.: 15/750,449

(22) PCT Filed: Aug. 1, 2016
(86) PCT No.: PCT/EP2016/068301

§ 371 Date: Feb. 5, 2018

(30) Foreign Application Priority Data
Aug. 7, 2015 (EP) 15180277.4

(51) Int. Cl. C07D 215/38; C07D 215/227; C07C 211/52; C07C 229/44; C07C 205/12

(52) U.S. Cl. CPC C07D 215/38; C07D 215/227; C07C 205/12; C07C 229/44; C07C 211/52

(57) ABSTRACT

The present invention relates to a novel process for the preparation of 5-Amino-quinolin-2(1H)-ones and its tautomer's 5-amino-quinolin-2-ols. The present invention further comprises various novel compounds which are obtained during the preparation of 5-Amino-quinolin-2(1H)-ones and its tautomer's 5-amino-quinolin-2-ols.

The present invention relates to a novel process for the preparation of 5-Amino-quinolin-2(1H)-ones and its tautomer's 5-amino-quinolin-2-ols.

5-Amino-quinolin-2(1H)-ones as such are already known in the art. 5-Amino-quinolin-2(1H)-ones are known as substituent for benzyl amine compounds which show anti-inflammatory efficiency and which are used as anti-inflammatory agents (cf. WO 2008/006627 A1).

5-Amino-quinolin-2H(1H)-one compounds of the general formula (I)

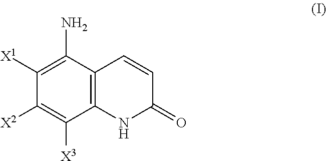

in which

X¹, X² and X³ are identical or different and independently selected from the group consisting of H, F and Cl, can be prepared in a variety of ways.

For example, in one known process (cf. WO 2009/065503 A1), in a first step, 2-bromo-3-fluoroaniline (A) is reacted with cinnamoyl chloride in the presence of pyridine and dichloromethane. The resulting N-(2-bromo-3-fluorophenyl)-3-phenylarylamide (B) is cyclized in the presence of AlCl₃ and benzene is eliminated to give 8-bromo-7-fluoro-1H-quinolin-2-one (C). The carbonyl group of (C) is protected in the next step by reacting (C) with POCl₃ to obtain 8-bromo-2-chloro-7-fluoroquinoline (D), which is subsequently nitrated by fuming HNO₃ (100%) in oleum (fuming sulfuric acid). The resulting 8-bromo-7-fluoro-5-nitroquinoline (E) is reacted with a mixture of HOAc and conc. HCl to deprotect the carbonyl group resulting in 8-bromo-7-fluoro-5-nitro-1H-quinolin-2-one (F). The resultant compound (F) is then reduced with Pd/C and ammonium formate to give finally a 5-amino-quinolin-2(1H)-one compound according to formula (I), in this case 5-amino-7-fluoro-quinolin-1H-one:

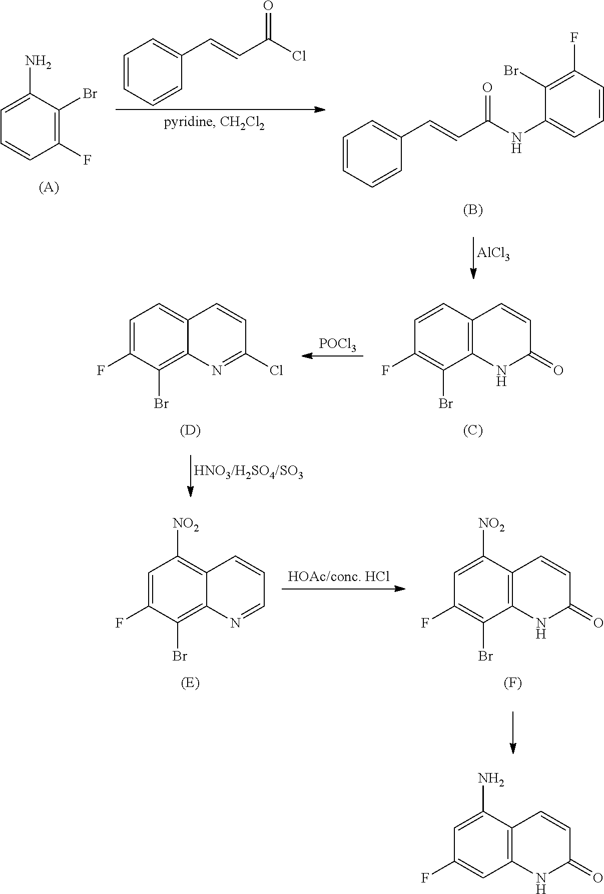

A disadvantage of this process is the relative high number of reaction steps resulting in a higher technical effort and higher production costs. Furthermore, the known process is very time-consuming. Additionally two of the reactions (cyclisation and nitration) proceed under very harsh conditions and therefore are as well accompanied with higher technical und safety effort. Finally, the reaction sequence starts with an expensive starting material which itself has to be prepared by a several step sequence.

As a consequence, there continues to be a need for processes for the synthesis of 5-amino-quinolin-2(1H)-one compounds of formula (I) which are technically simpler and more cost-effective.

It was therefore the object of this invention to provide a process for the preparation of 5-amino-quinolin-2(1H)-one compounds of formula (I) which produces the compound with a high yield and high purity and which can be carried out easily and cost-effective.

A new process has been found for the preparation of a 5-amino-quinolin-2(1H)-one compounds which avoids the aforementioned disadvantages. Furthermore, it can be carried out easily and cost-effectively, in particular due to the fact that the 5-amino-quinolin-2(1H)-one compounds according to the invention are obtained with high yields and in high purity from a cheap, readily available starting material.

The present invention provides a process for the preparation of a 5-amino-quinolin-2(1H)-one compound of the general formula (I)

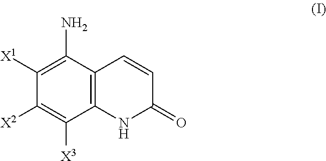

in which X¹, X² and X³ are identical or different and independently selected from the group consisting of H, F and Cl; and R¹ and R² may be same or different and are independently selected from the group consisting of H, C₁-C₁₂-alkyl, C₁-C₁₂-haloalkyl or halogen atoms.

Said compound is either produced by route (A), reduction of the nitro-group of a compound of the general formula (II)

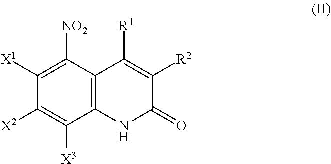

in which X¹, X², X³, R¹ and R² are as defined in formula (I)

or by

route (B), Heck reaction and in-situ cyclisation of a compound of the general formula (III),

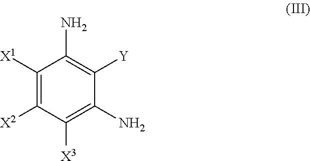

in which X¹, X² and X³ are as defined in formula (I), and Y is selected from Cl or Br, with the proviso that Y═Cl or Br when X¹, X² and X³═F, H or Y═Br when one of X¹, X² or X³═Cl with an acrylate compound of the general formula (IV)

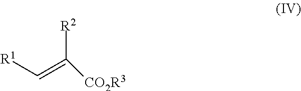

in which R¹ and R² are as defined in formula (I); and R³ is selected from the group consisting of hydrogen, substituted or unsubstituted C₁-C₁₂-alkyl, substituted or unsubstituted C₂-C₁₂-alkenyl, substituted or unsubstituted C₃-C₈-cycloalkyl, substituted or unsubstituted C₁-C₁₂-alkoxy, alkylaryl and aryl,

or any salt thereof

or by

route (C), cyclisation of a compound of the general formula (V)

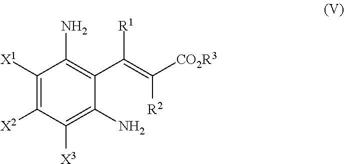

in which X¹, X², X³, R¹ and R² are as defined in formula (I), and R³ is as defined in formula (IV) in presence of a activation agent.

5-Amino-quinolin-2(1H)-ones can be prepared by using one of the routes (A), (B) or (C). The designation (A), (B) or (C) does not show a preference for one of these routes.

The process for the preparation of 5-amino-quinolin-2(1H)-one compound of the general formula (I) according to the present invention may also encompass every rotamer, tautomer and stereoisomer (cis/trans isomer) of the compounds of the present invention and mixtures thereof, in particular the tautomers of the compounds of the general formula (I) according to following general formula (Ia)

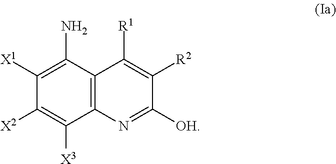

In the following, the specific routes (A), (B) and (C) are described in detail.

Route A

Step (A1)

The process for the preparation of a 5-amino-quinolin-2(1H)-one compound according to route (A) is a reduction of the nitro group of a compound of formula (II). The reduction of the nitro group is preferably selected from a metal reduction under acidic conditions, a reduction with sulfides or a catalytic hydrogenation.

The metal reduction under acidic is preferably carried out in the presence of at least one reducing agent of the group consisting of a metal, preferably selected from Fe, Sn or Zn, in combination with an acid, preferably acetic acid (HOAc), trifluoro acetic acid, hydrochloric acid (HCl), phosphoric acid or sulfuric acid, or combinations of metals, preferably selected from Fe or Zn in combination with salts selected from ammonium chloride, calcium chloride, iron (III) chloride. The reduction with sulfides is preferably carried out by using a sulfide, preferably H₂S together with a base (e.g. NaOH, NH₄OH), Na₂S, NaHS, Na₂S₂, (NH₄)₂S, NH₄HS, (NH₄)₂S₂ or mixtures of the aforementioned salts with sulfur (generating polysulfides), or other inorganic reducing agents, preferably sodium dithionite (Na₂S₂O₄), sodium bisulfite (NaHSO₃) or tin(II)chloride.

The amount of the reducing agent in the metal reduction used to the compound of formula (II) (reducing agent: compound of formula (II)) can vary. Preferably the molar ratio is in the range 1:1 to 10:1, preferably 1:1 to 5:1.

The amount of the reducing agent in the reduction with sulfides used to the compound of formula (II) (reducing agent: compound of formula (II)) can vary. Preferably the molar ratio is in the range 1:1 to 10:1, preferably 1:1 to 5:1.

The reduction in step (A1) of the compound of the general formula (II) according to the present invention can be carried out in an organic solvent or water. The solvent is preferably used in an amount such that the reaction mixture is readily stirrable during the entire process. The solvent is preferably inert under the reaction conditions. According to the invention, solvents are also understood as meaning mixtures of solvents, preferably pure solvents.

The organic solvents suitable for this reaction can be selected from the group consisting of alcohols (e.g. methanol, ethanol, isopropanol, butanol), ethers (e.g. methyl tert-butyl ether, n-butyl ether, anisole, tetrahydrofuran, dioxane, and polyethers of ethylene oxide and/or propylene oxide), aliphatic, cycloaliphatic or aromatic hydrocarbons (e.g. pentane, hexane, heptane, octane, nonane, and technical-grade hydrocarbons which may be substituted), esters (e.g. methyl acetate, ethyl acetate, butyl acetate, isobutyl acetate, and also dimethyl carbonate, dibutyl carbonate or ethylene carbonate), polar aprotic solvents (e.g. N,N-dimethylformamid, N,N-dimethylacetamid, acetonitrile, propionitril). Another solvent is water.

The reduction in step (A1) of a compound of the general formula (II) according to the invention can generally be carried out in vacuum, at atmospheric pressure or under superatmospheric pressure. Preferably the reaction is carried out at atmospheric pressure.

The temperatures applied during this process step can vary depending on the specific compound of formula (II) used for the reduction. The reduction in route (A) of a compound of the general formula (II) according to the present invention can be carried out at temperatures in the range of 0° C. to 150° C. The preferred temperature range for the reaction is between 20° C. and 100° C.

The reduction of the nitro group to an amino group can also be carried out by catalytic hydrogenation. Suitable catalysts to be used for the catalytic hydrogenation of route (A) comprise one or more metals of groups 8 to 10 of the Periodic Table, especially one or more metals selected from iron, ruthenium, osmium, cobalt, rhodium, iridium, nickel, palladium and platinum. Besides their catalytic activity, suitable catalysts may be inert under the selected reaction conditions.

The metals may be present in any chemical form, for example in elemental, colloidal, salt or oxide form, in combination with complexing agents such as chelates, or as alloys, in which case the alloys may also include other metals, for example aluminium, as well as the metals listed above.

The metals may be present in supported form, i.e. applied to any support, preferably an inorganic support. Examples of suitable supports are carbon (charcoal or activated carbon), aluminium oxide, silicon dioxide, zirconium dioxide or titanium dioxide. Catalysts preferred in accordance with the invention contain one or more metals of groups 8 to 10 of the Periodic Table on an inorganic support. Particular preference is given in accordance with the invention to catalysts which include palladium and platinum, and are optionally applied to an inorganic support (e.g. carbon). Such catalysts are, for example, platinum on carbon, platinum oxide on carbon and palladium on carbon.

In the catalytic hydrogenation according to the invention, the catalyst is used in an amount of about 0.01 to about 30% by weight based on compound of formula (II). The catalyst is preferably used in a concentration of about 0.1 to about 15% by weight.

The catalytic hydrogenation can be performed under elevated pressure (i.e. up to about 200 bar) in an autoclave, or at standard pressure in a hydrogen gas atmosphere. Especially at high reaction temperatures, it may be helpful to work at elevated pressure. The (additional) pressure increase can be brought about by supply of an inert gas, such as nitrogen or argon. The inventive catalytic hydrogenation is effected preferably at a pressure in the range from about 1 to about 30 bar, more preferably at a pressure in the range from about 5 to about 25 bar.

It is generally advantageous to perform the catalytic hydrogenation in the presence of solvents (diluents). However, the catalytic hydrogenation can also be performed without a solvent. Solvents are advantageously used in such an amount that the reaction mixture can efficiently be stirred over the entire process. Advantageously, based on compound (II) used, 1 to 50 times the amount of solvent, preferably 2 to 40 times the amount of solvent and more preferably 2 to 30 times the amount of solvent is used.

Useful solvents for performance of catalytic hydrogenation according to the present invention include all organic solvents which are inert under the reaction conditions. The solvent used depends on the type of reaction procedure, more particularly on the type of catalyst used and/or the hydrogen source (introduction of gaseous hydrogen or generation in situ). Mixtures of solvents can also be used.

Solvents suitable for the catalytic hydrogenation are halohydrocarbons, e.g. chlorohydrocarbons, such as tetrachloroethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene; alcohols such as methanol, ethanol, isopropanol, butanol; ethers, such as ethyl propyl ether, methyl tert-butyl ether, n-butyl ether, anisole, phenetole, cyclohexyl methyl ether, dimethyl ether, diethyl ether, dimethylglycol, diphenyl ether, dipropyl ether, diisopropyl ether, di-n-butyl ether, diisobutyl ether, diisoamyl ether, ethylene glycol dimethyl ether, isopropyl ethyl ether, methyl tert-butyl ether, tetrahydrofuran, dioxane, dichlorodiethyl ether, and polyethers of ethylene oxide and/or propylene oxide; aliphatic, cycloaliphatic or aromatic hydrocarbons such as pentane, hexane, heptane, octane, nonane, and technical-grade hydrocarbons which may be substituted by fluorine and chlorine atoms, such as methylene chloride, dichloromethane, trichloromethane, carbon tetrachloride, fluorobenzene, chlorobenzene or dichlorobenzene; for example white spirits having components with boiling points in the range, for example, from 40° C. to 250° C., cymene, petroleum fractions within a boiling range from 70° C. to 190° C., cyclohexane, methylcyclohexane, petroleum ether, ligroin, octane, benzene, toluene, chlorobenzene, bromobenzene, xylene; esters such as methyl acetate, ethyl acetate, butyl acetate, isobutyl acetate, and also dimethyl carbonate, dibutyl carbonate or ethylene carbonate. Another solvent is water.

The catalytic hydrogenation can optionally be performed in the presence of acids or bases. Acids suitable for the catalytic hydrogenation are inorganic acids, such as hydrochloric acid, hydrobromic acid, sulphuric acid, phosphoric acid; organic acids, such as acetic acid, trichloro acetic acid, trifluoro acetic acid and benzoic acid. Bases suitable for the catalytic hydrogenation are inorganic bases, e.g. alkali metal carbonates, such as sodium carbonate, potassium carbonate; alkaline earth metal carbonates, such as calcium carbonate; organic bases, e.g. alkylamines, such as triethylamine and ethyl di-iso-propyl amine.

In the catalytic hydrogenation according to the invention, the solvents used are preferably ethers, water or alcohols.

The catalytic hydrogenation according can be performed within a wide temperature range (for example in the range from about −20° C. to about 100° C.). Preference is given to performing the catalytic hydrogenation within a temperature range from about 0° C. to about 100° C., in particular room temperature (i.e. around 20° C.).

After the end of the reaction the reduction agent or the catalyst can be removed by filtration. It can be advantageous to use a filter aid such as Celite® in the filtration.

If the reduction or the catalytic hydrogenation is carried out in a solvent, the solvent can be removed by distillation or addition of water and extraction of the product into an organic solvent such as ethyl acetate, tert-butyl methyl ether and dichloromethane.

Step (A2)

The compound of the general formula (II) according to the present invention can be prepared by the cyclisation of a compound of the general formula (VI)

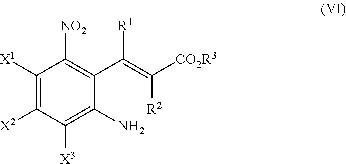

in which X¹, X², X³, R¹ and R² are as defined in formula (I); and R³ is as defined in formula (IV) in the presence of an activation agent.

The activation agent for the cyclisation reaction of the compound of the general formula (VI) can be selected from the group consisting of acids and bases, preferably bases.

Suitable bases include alkali metal salts (e.g. sodium carbonate, potassium carbonate, lithium hydride, sodium hydride, potassium hydride, butyl lithium, tert-butyl lithium, trimethylsilyl lithium, lithium hexamethyldisilazide, cesium carbonate, tripotassium phosphate, sodium acetate, potassium acetate, sodium methoxide, sodium ethoxide, sodium-tert-butoxide and potassium-tert-butoxide), organic bases (e.g. triethylamine, diisopropylethylamine, tributylamine, N-methylmorpholine, N,N-dimethylaniline, N,N-diethylaniline, 4-tert-butyl-N,N-dimethylaniline, 4-tert-butyl-N,N-diethylaniline, pyridine, picoline, lutidine, diazabicyclooctan (DABCO), diazabicyclo-nonen (DBN), diazabicycloundecen (DBU) and imidazole). Preferred bases are sodium carbonate, potassium carbonate, and sodium methoxide.

Suitable acids include inorganic acids (e.g. hydrochloric acid, hydrobromic acid. sulfuric acid, phosphoric acid, poly phosphoric acid, nitric acid) and organic acids (acetic acid, trifluoro acetic acid, methane sulfonic acid, para-toluene sulfonic acid, camphorsulfonic acid). Preferred acids are acetic acid and hydrochloric acid.

The molar ratio of the activation agent used to the compound of the general formula (VI) (activation agent: compound of formula (IV)) can vary. Preferably the molar ratio is in the range of 1:1 to 5:1.

The cyclisation reaction of step (A2) according to the present invention can be carried out in an organic solvent. The solvent is preferably used in an amount such that the reaction mixture is readily stirrable during the entire process. The solvent is preferably inert under the reaction conditions. According to the present invention, solvents are also understood as meaning mixtures of solvents, preferably pure solvents.

The organic solvents suitable for this reaction can be selected from the group consisting of alcohols (e.g. methanol, ethanol, propanol, iso-propanol, butanol) and aprotic solvents (e.g. acetonitrile, propionitrile, N,N-dimethylformamide, N,N-dimethyl acetamide)

The cyclisation reaction of route (A) according to the present invention can generally be carried out in vacuum, at atmospheric pressure or under superatmospheric pressure. Preferrably the reaction is carried out at atmospheric pressure.

The temperatures can vary depending on the specific compound of the general formula (VI) used for the cyclisation reaction. The cyclisation reaction of route (A) according to the invention can be carried out at temperatures in the range of 0° C. to 160° C.

Step (A3 a)

The compound of the general formula (VI) can be produced by a Heck reaction of a compound of the general formula (VII)

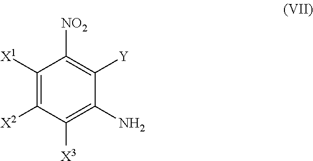

in which X¹, X² and X³ are as defined in formula (I) with the proviso that Y═Cl or Br when X¹, X² and X³═F, H or Y═Br when one of X¹, X² or X³═Cl; with an acrylate compound of the general formula (IV)

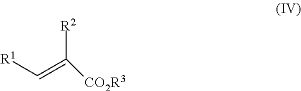

in which R¹ and R² are as defined in formula (I); and R³ is selected from the group consisting of hydrogen, substituted or unsubstituted C₁-C₁₂-alkyl, substituted or unsubstituted C₂-C₁₂-alkenyl, substituted or unsubstituted C₃-C₈-cycloalkyl, substituted or unsubstituted C₁-C₁₂-alkoxy, alkylaryl and aryl, or any salt thereof.

The acrylate compound within the Heck reaction of step (A3 a) according to the present invention can be selected from the group consisting of acrylic acids and its salts (e.g. sodium acrylate, potassium acrylate), methyl acrylate, ethyl acrylate, propyl acrylate, iso-propyl acrylate, butyl acrylate, phenyl acrylate and benzyl acrylate.

The stoichiometry of the compounds of the general formulae (VII) and (IV) used can vary within wide ranges. The molar ratio of the compound of the general formula (VII) to the acrylate compound of the general formula (IV) (compound of formula (VII): compound of formula (IV)) used can be in the range of 1:1 to 1:10.

A Heck reaction is a C—C coupling reaction with the use of a catalyst. The Heck reaction in step (A3 a) according to the present invention can be catalyzed by different palladium catalysts.

A palladium catalyst contains a palladium source and a ligand. The palladium catalyst can be formed in situ or be added as a preformed catalyst system. It is also possible to perform Heck reaction using a palladium source and e.g. the base as ligand (known as phosphine free Heck reaction).

The palladium source can be selected from the group consisting of palladium(II)acetate, palladium(II) chloride, palladium(II) bromide, tris(dibenzylidene-acetone)dipalladium(0), Tris(dibenzylideneacetone)dipalladium-chloroform adduct, bis(dibenzylideneacetone)palladium(0), bis(acetonitrile)palladium(II) chloride, and allylpalladium chloride dimer.

The ligand can be selected from the group consisting of aryl and heteroaryl phosphines (e.g. triphenylphosphine, tri-ortho-tolylphosphine, trimesitylphosphine, tri-(2-furyl)phosphine, 2-(dicyclohexylphosphino)-2′-isopropylbiphenyl, 2-(Dicyclohexylphosphino)-2′,4′,6′-triisopropylbiphenyl, 2-(di-tert-butylphosphino) biphenyl), 2-(dicyclohexylphosphino)biphenyl, 2-dicyclohexylphosphino-2′-(N,Ndimethylamino) biphenyl, 2-diphenylphosphino-2′-(N,N-dimethylamino) biphenyl, 2-(dicyclohexylphosphino)-2′-methylbiphenyl, 2-(di-tert-butylphosphino)-2′-methylbiphenyl, 2-di-tert-butylphosphino-2′-(N,Ndimethylamino) biphenyl, 2-dicyclo-hexylphosphino-2′,6′-diisopropoxy-1,1′-biphenyl, 2-Di-tert-butylphosphino-2′,4′,6′-triisopropylbiphenyl), bidentate phosphines (e.g. (+)-2,2′-bis(diphenylphosphino)-1,1′-binaphthyl, bis(diphenylphosphino)methane, 1,2-bis(diphenylphosphino)ethane, 1,2-bis(dicyclohexylphosphino)ethane, 1,3-bis(diphenylphosphino)propane, 1,3-bis(dicyclohexylphosphino)propane, 1,4-bis(diphenylphosphino)butane, 1,5-bis(diphenylphosphino)pentane, bis(2-diphenylphosphinophenyl)ether, 1,1′-bis(di-phenylphosphino)ferrocene, 1,1′-bis(diisopropylphosphino)ferrocene, 1,1′-bis(di-tert-butylphosphino)ferrocene, 1,2-bis(diphenylphosphino)benzene), alkyl phosphines (e.g. tricyclohexylphosphine, triisopropylphosphine, tri-n-butylphosphine, di-tert-butylmethylphosphine, tri-tert-butylphosphine) and their salts (e.g. tri-tert-butylphosphine tetrafluorborate, tricyclohexylphosphine tetrafluorborate), phosphites (e.g. trimethyl phosphite, triethyl phosphite, tri-iso-propyl phosphite, tricyclohexyl phosphite, triphenyl phosphite, tri-2,4-tert-butyl phosphite), N-heterocyclic carbene precursors (e.g. 1,3-bis(2,4,6-trimethylphenyl)imidazolium chloride, 1,3-bis(2,6-diisopropylphenyl)imidazolium chloride, 1,3-bis(adamant-1-yl)imidazolium chloride, 1,3-bis(2,4,6-trimethylphenyl)-4,5-dihydroimidazolium tetrafluoroborate, 1,3-bis(2,6-diisopropylphenyl) imidazolidinium tetrafluoroborate, 1,3-bis(2,4,6-trimethylphenyl)imidazolidinium chloride and 1,3-bis(2,6-diisopropylphenyl)imidazolidinium chloride).

The preformed catalyst can be selected from the group consisting of palladium phosphine catalysts (e.g. bis(tri-tert-butylphosphine) palladium (0), [1,2-bis(diphenyl-phosphino)ethane]dichloropalladium(II), 1,1′-bis (diphenylphosphino)ferrocene-palladium(II)dichloride dichloromethane adduct, bis(tricyclohexylphosphine)-palladium(0), bis(triethylphosphine)palladium(II) chloride, bis(triphenylphosphine)-palladium(II) acetate, bis(triphenylphosphine)palladium(II) chloride, bis(tri-t-butylphosphine)palladium(0), bis[1,2-bis(diphenylphosphino)ethane]palladium(0), bis[tri(o-tolyl)phosphine]palladium(II)chloride, dichlorobis(tricyclohexyl-phosphine)palladium(II), tetrakis(triphenylphosphine)palladium(0) and trans-benzyl-(chloro)bis(triphenylphosphine)palladium(II)).

Additionally preformed catalysts might be prepared from the above palladium sources and ligands either in a separate reaction or in situ before the other reaction partners are added.

Usually, the Heck reaction of step (A3 a) of the present invention can be carried out in the presence of an additive. Suitable additives include organic ammonium salts (e.g. tetrabutylammonium bromid, tetrabutylammonium chloride, tetrabuthylammonium bisulfate, tetrabutylammonium acetate, tetraethylammonium chlorid, tetraethylammonium bromid, benzyl trimethylammonium chlorid and benzyl trimethylammonium bromid), organic phosphonium salts (e.g. tetra-n-butylphosphoniumbromide) alkali and earth alkali salts (e.g. lithium chloride, lithium bromide, sodium chloride and magnesium chloride).

The Heck reaction of route (A) of the present invention can also be carried out in the presence of a base. Suitable bases include alkali metal salts (e.g. sodium carbonate, sodium bicarbonate, potassium carbonate, potassium bicarbonate, cesium carbonate, tripotassium phosphate, sodium acetate, potassium acetate, sodium methoxide, sodium ethoxide, sodium-tert-butoxide and potassium-tert-butoxide), organic bases (e.g. triethylamine, diisopropylethylamine, dicyclohexyl methylamine, tributylamine, N-methylmorpholine, N,N-dimethylaniline, N,N-diethylaniline, 4-tert-butyl-N,N-dimethylaniline, 4-tert-butyl-N,N-diethylaniline, pyridine, picoline, lutidine, and imidazole).

The molar ratio of the base or additive to the compound of formula (VII) (base or additive: compound of formula (VII)) can vary. Preferably the molar ratio is in the range of 1:1 to 5:1. Preferred is a molar ration in the range of 1:1 to 2:1.

The Heck reaction of route (A) according to the invention can be carried out in an aprotic organic solvent. The solvent is preferably used in an amount such that the reaction mixture is readily stirrable during the entire process. The solvent is preferably inert under the reaction conditions. According to the invention, solvents are also understood as meaning mixtures of solvents, preferably pure solvents.

The organic solvents suitable for this reaction can be selected from the group consisting of halohydrocarbons (e.g. chlorohydrocarbons, such as methylene chloride, 1,2-dichloroethane, chlorobenzene, bromobenzene, dichlorobenzene, chlorotoluene), alcohols (e.g. methanol, ethanol, isopropanol, butanol), ethers (e.g. methyl tert-butyl ether, n-butyl ether, anisole, tetrahydrofuran, dioxane, and polyethers of ethylene oxide and/or propylene oxide), aliphatic, cycloaliphatic or aromatic hydrocarbons (e.g. pentane, hexane, heptane, octane, nonane, and technical-grade hydrocarbons which may be substituted), esters (e.g. methyl acetate, ethyl acetate, butyl acetate, isobutyl acetate, and also dimethyl carbonate, dibutyl carbonate or ethylene carbonate) and polar aprotic solvents (e.g. N,N-dimethylformamid, N,N-dimethylacetamid, acetonitrile, propionitril). Another solvent is water. Preferably the reaction is run in butyl acetate.

The Heck reaction of route (A) according to the invention can generally be carried out in vacuum, at atmospheric pressure or under superatmospheric pressure. Preferrably the reaction is conducted at atmospheric pressure.

The temperatures can vary depending on the specific compounds of the general formulae (VII) and (IV) used for the Heck reaction. The Heck reaction of route (A) according to the present invention can be carried out preferably at temperatures in the range of 20° C. to 160° C.

Step (A3 b)

Alternatively, the compound of the general formula (VI) can be produced by the reduction of one of the nitro-groups of a compound of the general formula (IX)

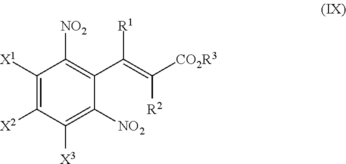

in which X¹, X², X³, R¹ and R² are as defined in formula (I), and R³ is as defined in formula (IV).

The reduction of the nitro group of the compound of the general formula (IX) in step (A3 b) is preferably selected from a metal reduction under acidic conditions, a reduction with sulfides or a catalytic hydrogenation.

The metal reduction under acidic is preferably carried out in the presence of at least one reducing agent of the group consisting of a metal, preferably selected from Fe, Sn or Zn, in combination with an acid, preferably acetic acid (HOAc), trifluoro acetic acid, hydrochloric acid (HCl), phosphoric acid or sulfuric acid, or combinations of metals, preferably selected from Fe or Zn in combination with salts selected from ammonium chloride, calcium chloride, iron (III) chloride. The reduction with sulfides is preferably carried out by using a sulfide, preferably H₂S together with a base (e.g. NaOH, NH₄OH), Na₂S, NaHS, Na₂S₂, (NH₄)₂S, NH₄HS, (NH₄)₂S₂ or mixtures of the aforementioned salts with sulfur (generating polysulfides), or other inorganic reducing agents, preferably sodium dithionite (Na₂S₂O₄), sodium bisulfite (NaHSO₃) or tin(II)chloride.

The amount of the reducing agent in the metal reduction used to the compound of formula (IX) (reducing agent: compound of formula (IX)) can vary. Preferably the molar ratio is in the range 1:1 to 10:1, preferably 1:1 to 5:1.

The amount of the reducing agent in the reduction with sulfides used to the compound of formula (IX) (reducing agent: compound of formula (IX)) can vary. Preferably the molar ratio is in the range 1:1 to 10:1, preferably 1:1 to 5:1.

The reduction in step (A3 b) of the compound of the general formula (IX) according to the present invention can be carried out in an organic solvent or water. The solvent is preferably used in an amount such that the reaction mixture is readily stirrable during the entire process. The solvent is preferably inert under the reaction conditions. According to the invention, solvents are also understood as meaning mixtures of solvents, preferably pure solvents.

The organic solvents suitable for this reaction can be selected from the group consisting of alcohols (e.g. methanol, ethanol, isopropanol, butanol), ethers (e.g. methyl tert-butyl ether, n-butyl ether, anisole, tetrahydrofuran, dioxane, and polyethers of ethylene oxide and/or propylene oxide), aliphatic, cycloaliphatic or aromatic hydrocarbons (e.g. pentane, hexane, heptane, octane, nonane, and technical-grade hydrocarbons which may be substituted), esters (e.g. methyl acetate, ethyl acetate, butyl acetate, isobutyl acetate, and also dimethyl carbonate, dibutyl carbonate or ethylene carbonate), polar aprotic solvents (e.g. N,N-dimethylformamid, N,N-dimethylacetamid, acetonitrile, propionitril). Another solvent is water.

The reduction of step (A3 b) of a compound of the general formula (IX) according to the invention can generally be carried out in vacuum, at atmospheric pressure or under superatmospheric pressure. Preferably the reaction is carried out at atmospheric pressure.

The temperatures applied during this process step can vary depending on the specific compound of formula (IX) used for the reduction. The reduction in route (A) of a compound of the general formula (IX) according to the present invention can be carried out at temperatures in the range of 0° C. to 150° C. The preferred temperature range for the reaction is between 20° C. and 100° C.

The reduction of the nitro group to an amino group can also be carried out by catalytic hydrogenation. Suitable catalysts to be used for the catalytic hydrogenation of route (A) comprise one or more metals of groups 8 to 10 of the Periodic Table, especially one or more metals selected from iron, ruthenium, osmium, cobalt, rhodium, iridium, nickel, palladium and platinum. Besides their catalytic activity, suitable catalysts may be inert under the selected reaction conditions.

The metals may be present in any chemical form, for example in elemental, colloidal, salt or oxide form, in combination with complexing agents such as chelates, or as alloys, in which case the alloys may also include other metals, for example aluminium, as well as the metals listed above.

The metals may be present in supported form, i.e. applied to any support, preferably an inorganic support. Examples of suitable supports are carbon (charcoal or activated carbon), aluminium oxide, silicon dioxide, zirconium dioxide or titanium dioxide. Catalysts preferred in accordance with the invention contain one or more metals of groups 8 to 10 of the Periodic Table on an inorganic support. Particular preference is given in accordance with the invention to catalysts which include palladium and platinum, and are optionally applied to an inorganic support (e.g. carbon). Such catalysts are, for example, platinum on carbon, platinum oxide on carbon and palladium on carbon.

In the catalytic hydrogenation according to the invention, the catalyst is used in an amount of about 0.01 to about 30% by weight based on compound of formula (IX). The catalyst is preferably used in a concentration of about 0.1 to about 15% by weight.

The catalytic hydrogenation can be performed under elevated pressure (i.e. up to about 200 bar) in an autoclave, or at standard pressure in a hydrogen gas atmosphere. Especially at high reaction temperatures, it may be helpful to work at elevated pressure. The (additional) pressure increase can be brought about by supply of an inert gas, such as nitrogen or argon. The inventive catalytic hydrogenation is effected preferably at a pressure in the range from about 1 to about 30 bar, more preferably at a pressure in the range from about 5 to about 25 bar.

It is generally advantageous to perform the catalytic hydrogenation in the presence of solvents (diluents). However, the catalytic hydrogenation can also be performed without a solvent. Solvents are advantageously used in such an amount that the reaction mixture can efficiently be stirred over the entire process. Advantageously, based on compound (IX) used, 1 to 50 times the amount of solvent, preferably 2 to 40 times the amount of solvent and more preferably 2 to 30 times the amount of solvent is used.

Useful solvents for performance of catalytic hydrogenation according to the present invention include all organic solvents which are inert under the reaction conditions. The solvent used depends on the type of reaction procedure, more particularly on the type of catalyst used and/or the hydrogen source (introduction of gaseous hydrogen or generation in situ). Mixtures of solvents can also be used.

Solvents suitable for the catalytic hydrogenation are halohydrocarbons, e.g. chlorohydrocarbons, such as tetrachloroethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene; alcohols such as methanol, ethanol, isopropanol, butanol; ethers, such as ethyl propyl ether, methyl tert-butyl ether, n-butyl ether, anisole, phenetole, cyclohexyl methyl ether, dimethyl ether, diethyl ether, dimethylglycol, diphenyl ether, dipropyl ether, diisopropyl ether, di-n-butyl ether, diisobutyl ether, diisoamyl ether, ethylene glycol dimethyl ether, isopropyl ethyl ether, methyl tert-butyl ether, tetrahydrofuran, dioxane, dichlorodiethyl ether, and polyethers of ethylene oxide and/or propylene oxide; aliphatic, cycloaliphatic or aromatic hydrocarbons such as pentane, hexane, heptane, octane, nonane, and technical-grade hydrocarbons which may be substituted by fluorine and chlorine atoms, such as methylene chloride, dichloromethane, trichloromethane, carbon tetrachloride, fluorobenzene, chlorobenzene or dichlorobenzene; for example white spirits having components with boiling points in the range, for example, from 40° C. to 250° C., cymene, petroleum fractions within a boiling range from 70° C. to 190° C., cyclohexane, methylcyclohexane, petroleum ether, ligroin, octane, benzene, toluene, chlorobenzene, bromobenzene, xylene; esters such as methyl acetate, ethyl acetate, butyl acetate, isobutyl acetate, and also dimethyl carbonate, dibutyl carbonate or ethylene carbonate. Another solvent is water.

The catalytic hydrogenation can optionally be performed in the presence of acids or bases. Acids suitable for the catalytic hydrogenation are inorganic acids, such as hydrochloric acid, hydrobromic acid, sulphuric acid, phosphoric acid; organic acids, such as acitic acid, trichloro acetic acid, trifluoro acetic acid and benzoic acid. Bases suitable for the catalytic hydrogenation are inorganic bases, e.g. alkali metal carbonates, such as sodium carbonate, potassium carbonate; alkaline earth metal carbonates, such as calcium carbonate; organic bases, e.g. alkylamines, such as triethylamine and ethyl di-iso-propyl amine.

In the catalytic hydrogenation according to the invention, the solvents used are preferably ethers, water or alcohols.

The catalytic hydrogenation according can be performed within a wide temperature range (for example in the range from about −20° C. to about 100° C.). Preference is given to performing the catalytic hydrogenation within a temperature range from about 0° C. to about 100° C., in particular room temperature (i.e. around 20° C.).

After the end of the reaction the reduction agent or the catalyst can be removed by filtration. It can be advantageous to use a filter aid such as Celite® in the filtration.

If the reduction or the catalytic hydrogenation is carried out in a solvent, the solvent can be removed by distillation or addition of water and extraction of the product into an organic solvent such as ethyl acetate, tert-butyl methyl ether, dichloromethane.

Step (A4) The compound of the general formula (VII) can be prepared by the reduction of a compound of the general formula (VIII)

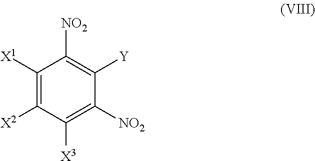

in which X¹, X² and X³ are as defined in formula (I) with the proviso that Y═Cl or Br when X¹, X² and X³═F, H or Y═Br when one of X¹, X² or X³═Cl.

The reduction of the nitro group of the compound of the general formula (VIII) in step (A4) is preferably selected from a metal reduction under acidic conditions, a reduction with sulfides or a catalytic hydrogenation.

The metal reduction under acidic is preferably carried out in the presence of at least one reducing agent of the group consisting of a metal, preferably selected from Fe, Sn or Zn, in combination with an acid, preferably acetic acid (HOAc), trifluoro acetic acid, hydrochloric acid (HCl), phosphoric acid or sulfuric acid, or combinations of metals, preferably selected from Fe or Zn in combination with salts selected from ammonium chloride, calcium chloride, iron (III) chloride. The reduction with sulfides is preferably carried out by using a sulfide, preferably H₂S together with a base (e.g. NaOH, NH₄OH), Na₂S, NaHS, Na₂S₂, (NH₄)₂S, NH₄HS, (NH₄)₂S₂ or mixtures of the aforementioned salts with sulfur (generating polysulfides), or other inorganic reducing agents, preferably sodium dithionite (Na₂S₂O₄), sodium bisulfite (NaHSO₃) or tin(II)chloride.

The amount of the reducing agent in the metal reduction used to the compound of formula (VIII) (reducing agent: compound of formula (VIII)) can vary. Preferably the molar ratio is in the range 1:1 to 10:1, preferably 1:1 to 5:1.

The amount of the reducing agent in the reduction with sulfides used to the compound of formula (VIII) (reducing agent: compound of formula (VIII)) can vary. Preferably the molar ratio is in the range 1:1 to 10:1, preferably 1:1 to 5:1.

The reduction of step (A4) of the compound of the general formula (VIII) according to the present invention can be carried out in an organic solvent. The solvent is preferably used in an amount such that the reaction mixture is readily stirrable during the entire process. The solvent is preferably inert under the reaction conditions. According to the invention, solvents are also understood as meaning mixtures of solvents, preferably pure solvents.

The organic solvents suitable for this reaction can be selected from the group consisting of alcohols (e.g. methanol, ethanol, isopropanol, butanol), ethers (e.g. methyl tert-butyl ether, n-butyl ether, anisole, tetrahydrofuran, dioxane, and polyethers of ethylene oxide and/or propylene oxide), aliphatic, cycloaliphatic or aromatic hydrocarbons (e.g. pentane, hexane, heptane, octane, nonane, and technical-grade hydrocarbons which may be substituted), esters (e.g. methyl acetate, ethyl acetate, butyl acetate, isobutyl acetate, and also dimethyl carbonate, dibutyl carbonate or ethylene carbonate), polar aprotic solvents (e.g. N,N-dimethylformamid, N,N-dimethylacetamid, acetonitrile, propionitril). Another solvent is water.

The reduction of step (A4) of a compound of the general formula (VIII) according to the present invention can generally be carried out in vacuum, at atmospheric pressure or under superatmospheric pressure. Preferrably the reaction is carried out at atmospheric pressure.

The temperatures applied during this process step can vary depending on the specific compound of formula (VIII) used for the reduction. The reduction in step (A4) of a compound of the general formula (VIII) according to the present invention can be carried out at temperatures in the range of 0° C. to 150° C. The preferred temperature range for the reaction is between 20° C. and 100° C.

The reduction of the nitro group to an amino group can also be carried out by catalytic hydrogenation. Suitable catalysts to be used for the catalytic hydrogenation in step (A4) of route (A) comprise one or more metals of groups 8 to 10 of the Periodic Table, especially one or more metals selected from iron, ruthenium, osmium, cobalt, rhodium, iridium, nickel, palladium and platinum. Besides their catalytic activity, suitable catalysts may be inert under the selected reaction conditions.

The metals may be present in any chemical form, for example in elemental, colloidal, salt or oxide form, in combination with complexing agents such as chelates, or as alloys, in which case the alloys may also include other metals, for example aluminium, as well as the metals listed above.

The metals may be present in supported form, i.e. applied to any support, preferably an inorganic support. Examples of suitable supports are carbon (charcoal or activated carbon), aluminium oxide, silicon dioxide, zirconium dioxide or titanium dioxide. Catalysts preferred in accordance with the invention contain one or more metals of groups 8 to 10 of the Periodic Table on an inorganic support. Particular preference is given in accordance with the invention to catalysts which include palladium and platinum, and are optionally applied to an inorganic support (e.g. carbon). Such catalysts are, for example, platinum on carbon, platinum oxide on carbon and palladium on carbon.

In the catalytic hydrogenation according to the invention, the catalyst is used in an amount of about 0.01 to about 30% by weight based on compound of formula (VIII). The catalyst is preferably used in a concentration of about 0.1 to about 15% by weight.

The catalytic hydrogenation can be performed under elevated pressure (i.e. up to about 200 bar) in an autoclave, or at standard pressure in a hydrogen gas atmosphere. Especially at high reaction temperatures, it may be helpful to work at elevated pressure. The (additional) pressure increase can be brought about by supply of an inert gas, such as nitrogen or argon. The inventive catalytic hydrogenation is effected preferably at a pressure in the range from about 1 to about 30 bar, more preferably at a pressure in the range from about 5 to about 25 bar.

It is generally advantageous to perform the catalytic hydrogenation in the presence of solvents (diluents). However, the catalytic hydrogenation can also be performed without a solvent. Solvents are advantageously used in such an amount that the reaction mixture can efficiently be stirred over the entire process. Advantageously, based on compound (VIII) used, 1 to 50 times the amount of solvent, preferably 2 to 40 times the amount of solvent and more preferably 2 to 30 times the amount of solvent is used.

Useful solvents for performance of catalytic hydrogenation according to the present invention include all organic solvents which are inert under the reaction conditions. The solvent used depends on the type of reaction procedure, more particularly on the type of catalyst used and/or the hydrogen source (introduction of gaseous hydrogen or generation in situ). Mixtures of solvents can also be used.

Solvents suitable for the catalytic hydrogenation are halohydrocarbons, e.g. chlorohydrocarbons, such as tetrachloroethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene; alcohols such as methanol, ethanol, isopropanol, butanol; ethers, such as ethyl propyl ether, methyl tert-butyl ether, n-butyl ether, anisole, phenetole, cyclohexyl methyl ether, dimethyl ether, diethyl ether, dimethylglycol, diphenyl ether, dipropyl ether, diisopropyl ether, di-n-butyl ether, diisobutyl ether, diisoamyl ether, ethylene glycol dimethyl ether, isopropyl ethyl ether, methyl tert-butyl ether, tetrahydrofuran, dioxane, dichlorodiethyl ether, and polyethers of ethylene oxide and/or propylene oxide; aliphatic, cycloaliphatic or aromatic hydrocarbons such as pentane, hexane, heptane, octane, nonane, and technical-grade hydrocarbons which may be substituted by fluorine and chlorine atoms, such as methylene chloride, dichloromethane, trichloromethane, carbon tetrachloride, fluorobenzene, chlorobenzene or dichlorobenzene; for example white spirits having components with boiling points in the range, for example, from 40° C. to 250° C., cymene, petroleum fractions within a boiling range from 70° C. to 190° C., cyclohexane, methylcyclohexane, petroleum ether, ligroin, octane, benzene, toluene, chlorobenzene, bromobenzene, xylene; esters such as methyl acetate, ethyl acetate, butyl acetate, isobutyl acetate, and also dimethyl carbonate, dibutyl carbonate or ethylene carbonate. Another solvent is water.

The catalytic hydrogenation can optionally be performed in the presence of acids or bases. Acids suitable for the catalytic hydrogenation are inorganic acids, such as hydrochloric acid, hydrobromic acid, sulphuric acid, phosphoric acid; organic acids, such as acitic acid, trichloro acetic acid, trifluoro acetic acid and benzoic acid. Bases suitable for the catalytic hydrogenation are inorganic bases, e.g. alkali metal carbonates, such as sodium carbonate, potassium carbonate; alkaline earth metal carbonates, such as calcium carbonate; organic bases, e.g. alkylamines, such as triethylamine and ethyl di-iso-propyl amine.

In the catalytic hydrogenation according to the invention, the solvents used are preferably ethers or alcohols.

The catalytic hydrogenation according can be performed within a wide temperature range (for example in the range from about −20° C. to about 100° C.). Preference is given to performing the catalytic hydrogenation within a temperature range from about 0° C. to about 100° C., in particular room temperature (i.e. around 20° C.).

Route B

Step (B1)

The process for the preparation of a 5-amino-quinolin-2(1H)-one compound according to route (B) is a Heck reaction and in-situ cyclisation of a compound of the general formula (III)

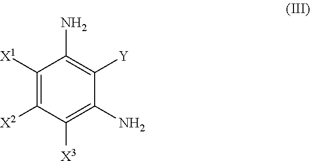

in which X¹, X² and X³ are as defined in formula (I), and Y is selected from Cl or Br, with the proviso that Y═Cl or Br when X¹, X² and X³═F, H or Y═Br when one of X¹, X² or X³═Cl; with an acrylate compound of the general formula (IV)

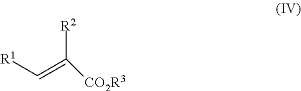

in which R¹ and R² are as defined in formula (I); and R³ is selected from the group consisting of hydrogen, substituted or unsubstituted C₁-C₁₂-alkyl, substituted or unsubstituted C₂-C₁₂-alkenyl, substituted or unsubstituted C₃-C₈-cycloalkyl, substituted or unsubstituted C₁-C₁₂-alkoxy, C₁-C₁₂-alkylaryl and aryl, or any salt thereof.

The acrylate compound within the Heck reaction in step (B1) according to the present invention can be selected from the group consisting of acrylic acids and its salts (e.g. sodium acrylate, potassium acrylate), methyl acrylate, ethyl acrylate, propyl acrylate, iso-propyl acrylate, butyl acrylate, phenyl acrylate and benzyl acrylate.

The stoichiometry of the compounds of the general formulae (III) and (IV) used can vary within wide ranges. The molar ratio of the compound of the general formula (III) to the acrylate compound of the general formula (IV) (compound of formula (III): compound of formula (IV)) used can be in the range of 1:1 to 1:10.

A Heck reaction is a C—C coupling reaction with the use of a catalyst. The Heck reaction of route (B) according to the present invention can be catalyzed by different palladium catalysts.

A palladium catalyst contains a palladium source and a ligand. The palladium catalyst can be formed in situ or be added as a preformed catalyst system. It is also possible to perform Heck reaction using a palladium source and e.g. the base as ligand (known as phosphine free Heck reaction).

The palladium source can be selected from the group consisting of palladium(II)acetate, palladium(II) chloride, palladium(II) bromide, tris(dibenzylidene-acetone)dipalladium(0), Tris(dibenzylideneacetone)dipalladium-chloroform adduct, bis(dibenzylideneacetone)palladium(0), bis(acetonitrile)palladium(II) chloride, and allylpalladium chloride dimer.

The ligand can be selected from the group consisting of aryl and heteroaryl phosphines (e.g. triphenylphosphine, tri-ortho-tolylphosphine, trimesitylphosphine, tri-(2-furyl)phosphine, 2-(dicyclohexylphosphino)-2′-isopropylbiphenyl, 2-(Dicyclohexylphosphino)-2′,4′,6′-triisopropylbiphenyl, 2-(di-tert-butylphosphino) biphenyl), 2-(dicyclohexylphosphino)biphenyl, 2-dicyclohexylphosphino-2′-(N,Ndimethylamino) biphenyl, 2-diphenylphosphino-2′-(N,N-dimethylamino) biphenyl, 2-(dicyclohexylphosphino)-2′-methylbiphenyl, 2-(di-tert-butylphosphino)-2′-methylbiphenyl, 2-di-tert-butylphosphino-2′-(N,Ndimethylamino) biphenyl, 2-dicyclo-hexylphosphino-2′,6′-diisopropoxy-1,1′-biphenyl, 2-Di-tert-butylphosphino-2′,4′,6′-triisopropylbiphenyl), bidentate phosphines (e.g. (+)-2,2′-bis(diphenylphosphino)-1,1′-binaphthyl, bis(diphenylphosphino)methane, 1,2-bis(diphenylphosphino)ethane, 1,2-bis(dicyclohexylphosphino)ethane, 1,3-bis(diphenylphosphino)propane, 1,3-bis(dicyclohexylphosphino)propane, 1,4-bis(diphenylphosphino)butane, 1,5-bis(diphenylphosphino)pentane, bis(2-diphenylphosphinophenyl)ether, 1,1′-bis(di-phenylphosphino)ferrocene, 1,1′-bis(diisopropylphosphino)ferrocene, 1,1′-bis(di-tert-butylphosphino)ferrocene, 1,2-bis(diphenylphosphino)benzene), alkyl phosphines (e.g. tricyclohexylphosphine, triisopropylphosphine, tri-n-butylphosphine, di-tert-butylmethylphosphine, tri-tert-butylphosphine) and their salts (e.g. tri-tert-butylphosphine tetrafluorborate, tricyclohexylphosphine tetrafluorborate), phosphites (e.g. trimethyl phosphite, triethyl phosphite, tri-iso-propyl phosphite, tricyclohexyl phosphite, triphenyl phosphite, tri-2,4-tert-butyl phosphite), N-heterocyclic carbene precursors (e.g. 1,3-bis(2,4,6-trimethylphenyl)imidazolium chloride, 1,3-bis(2,6-diisopropylphenyl)imidazolium chloride, 1,3-bis(adamant-1-yl)imidazolium chloride, 1,3-bis(2,4,6-trimethylphenyl)-4,5-dihydroimidazolium tetrafluoroborate, 1,3-bis(2,6-diisopropylphenyl) imidazolidinium tetrafluoroborate, 1,3-bis(2,4,6-trimethylphenyl)imidazolidinium chloride and 1,3-bis(2,6-diisopropylphenyl)imidazolidinium chloride).

The preformed catalyst can be selected from the group consisting of palladium phosphine catalysts (e.g. bis(tri-tert-butylphosphine) palladium (0), [1,2-bis(diphenyl-phosphino)ethane]dichloropalladium(II), 1,1′-bis (diphenylphosphino)ferrocene-palladium(II)dichloride dichloromethane adduct, bis(tricyclohexylphosphine)-palladium(0), bis(triethylphosphine)palladium(II) chloride, bis(triphenylphosphine)-palladium(II) acetate, bis(triphenylphosphine)palladium(II) chloride, bis(tri-t-butylphosphine)palladium(0), bis[1,2-bis(diphenylphosphino)ethane]palladium(0), bis[tri(o-tolyl)phosphine]palladium(II)chloride, dichlorobis(tricyclohexyl-phosphine)palladium(II), tetrakis(triphenylphosphine)palladium(0) and trans-benzyl-(chloro)bis(triphenylphosphine)palladium(II)).

Additionally preformed catalysts might be prepared from the above palladium sources and ligands either in a separate reaction or in situ before the other reaction partners are added.

Usually, the Heck reaction of route (B) of the present invention can be carried out in the presence of an additive. Suitable additives include organic ammonium salts (e.g. tetrabutylammonium bromid, tetrabutylammonium chloride, tetrabuthylammonium bisulfate, tetrabutylammonium acetate, tetraethylammonium chlorid, tetraethylammonium bromid, benzyl trimethylammonium chlorid and benzyl trimethylammonium bromid), organic phosphonium salts (e.g. tetra-n-butylphosphoniumbromide) alkali and earth alkali salts (e.g. lithium chloride, lithium bromide, sodium chloride and magnesium chloride).

The Heck reaction of route (B) of the present invention can also be carried out in the presence of a base. Suitable bases include alkali metal salts (e.g. sodium carbonate, sodium bicarbonate, potassium carbonate, potassium bicarbonate, cesium carbonate, tripotassium phosphate, sodium acetate, potassium acetate, sodium methoxide, sodium ethoxide, sodium-tert-butoxide and potassium-tert-butoxide), organic bases (e.g. triethylamine, diisopropylethylamine, dicyclohexyl methylamine, tributylamine, N-methylmorpholine, N,N-dimethylaniline, N,N-diethylaniline, 4-tert-butyl-N,N-dimethylaniline, 4-tert-butyl-N,N-diethylaniline, pyridine, picoline, lutidine, and imidazole).

The molar ratio of the base or additive to the compound of formula (III) (base or additive: compound of formula (III) can vary. Preferably the molar ratio is in the range of 1:1 to 5:1. Preferred is a molar ratio in the range of 1:1 to 2:1.

The Heck reaction of route (B) according to the invention can be carried out in an aprotic organic solvent. The solvent is preferably used in an amount such that the reaction mixture is readily stirrable during the entire process. The solvent is preferably inert under the reaction conditions. According to the invention, solvents are also understood as meaning mixtures of solvents, preferably pure solvents.

The organic solvents suitable for this reaction can be selected from the group consisting of halohydrocarbons (e.g. chlorohydrocarbons, such as methylene chloride, 1,2-dichloroethane, chlorobenzene, bromobenzene, dichlorobenzene, chlorotoluene), alcohols (e.g. methanol, ethanol, isopropanol, butanol), ethers (e.g. methyl tert-butyl ether, n-butyl ether, anisole, tetrahydrofuran, dioxane, and polyethers of ethylene oxide and/or propylene oxide), aliphatic, cycloaliphatic or aromatic hydrocarbons (e.g. pentane, hexane, heptane, octane, nonane, and technical-grade hydrocarbons which may be substituted), esters (e.g. methyl acetate, ethyl acetate, butyl acetate, isobutyl acetate, and also dimethyl carbonate, dibutyl carbonate or ethylene carbonate) and polar aprotic solvents (e.g. N,N-dimethylformamid, N,N-dimethylacetamid, acetonitrile, propionitril). Another solvent is water.

The Heck reaction of route (B) according to the invention can generally be carried out in vacuum, at atmospheric pressure or under superatmospheric pressure. Preferably the reaction is carried out at atmospheric pressure.

The temperatures can vary depending on the specific compounds of formulae (III) and (IV) used for the Heck reaction. The Heck reaction of route (B) according to the present invention can be carried out at temperatures in the range of 20° C. to 160° C. Preferably the reaction is carried out at temperatures in the range of 50° C. to 120° C.

In certain cases, depending on the catalyst (e.g. with (+)-2,2′-bis(diphenylphosphino)-1,1′-binaphthyl) the cyclisation cannot carried out under the basic Heck reaction conditions. Then it is advantageous to add acid (e.g. acetic acid) in an amount to neutralize the base and further acidifying the reaction to induce the cyclisation reaction under heating of the reaction mixture. After that the cyclized product can be isolated.

Step (B2)

The compound of the general formula (III) can be prepared by the reduction of both nitro groups of a compound of the general formula (VIII)

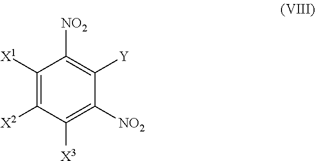

in which X¹, X² and X³ are as defined in formula (I) with the proviso that Y═Cl or Br when X¹, X² and X³═F, H or Y═Br when one of X¹, X² or X³═Cl.

The reduction of both nitro groups of the compound of the general formula (VIII) in step (B2) is preferably selected from a metal reduction under acidic conditions, a reduction with sulfides or a catalytic hydrogenation.

The metal reduction under acidic is preferably carried out in the presence of at least one reducing agent of the group consisting of a metal, preferably selected from Fe, Sn or Zn, in combination with an acid, preferably acetic acid (HOAc), trifluoro acetic acid, hydrochloric acid (HCl), phosphoric acid or sulfuric acid, or combinations of metals, preferably selected from Fe or Zn in combination with salts selected from ammonium chloride, calcium chloride, iron (III) chloride. The reduction with sulfides is preferably carried out by using a sulfide, preferably H₂S together with a base (e.g. NaOH, NH₄OH), Na₂S, NaHS, Na₂S₂, (NH₄)₂S, NH₄HS, (NH₄)₂S₂ or mixtures of the aforementioned salts with sulfur (generating polysulfides), or other inorganic reducing agents, preferably sodium dithionite (Na₂S₂O₄), sodium bisulfite (NaHSO₃) or tin(II)chloride.

The amount of the reducing agent in the metal reduction used to the compound of formula (VIII) (reducing agent: compound of formula (VIII)) can vary. Preferably the molar ratio is in the range 1:1 to 10:1, preferably 1:1 to 5:1.

The amount of the reducing agent in the reduction with sulfides used to the compound of formula (VIII) (reducing agent: compound of formula (VIII)) can vary. Preferably the molar ratio is in the range 1:1 to 10:1, preferably 1:1 to 5:1.

The reduction of step (B2) of the compound of the general formula (VIII) according to the present invention can be carried out in an organic solvent. The solvent is preferably used in an amount such that the reaction mixture is readily stirrable during the entire process. The solvent is preferably inert under the reaction conditions. According to the invention, solvents are also understood as meaning mixtures of solvents, preferably pure solvents.

The organic solvents suitable for this reaction can be selected from the group consisting of alcohols (e.g. methanol, ethanol, isopropanol, butanol), ethers (e.g. methyl tert-butyl ether, n-butyl ether, anisole, tetrahydrofuran, dioxane, and polyethers of ethylene oxide and/or propylene oxide), aliphatic, cycloaliphatic or aromatic hydrocarbons (e.g. pentane, hexane, heptane, octane, nonane, and technical-grade hydrocarbons which may be substituted), esters (e.g. methyl acetate, ethyl acetate, butyl acetate, isobutyl acetate, and also dimethyl carbonate, dibutyl carbonate or ethylene carbonate), polar aprotic solvents (e.g. N,N-dimethylformamid, N,N-dimethylacetamid, acetonitrile, propionitril). Another solvent is water.

The reduction of step (B2) of a compound of the general formula (VIII) according to the present invention can generally be carried out in vacuum, at atmospheric pressure or under superatmospheric pressure. Preferably the reaction is carried out at atmospheric pressure.

The temperatures applied during this process step can vary depending on the specific compound of formula (VIII) used for the reduction. The reduction in step (B2) of a compound of the general formula (VIII) according to the present invention can be carried out at temperatures in the range of 0° C. to 150° C. Preferably the reaction is carried out at temperatures in the range of 20° C. to 100° C.

The reduction of the nitro group to an amino group can also be carried out by catalytic hydrogenation. Suitable catalysts to be used for the catalytic hydrogenation in step (B2) of route (B) comprise one or more metals of groups 8 to 10 of the Periodic Table, especially one or more metals selected from iron, ruthenium, osmium, cobalt, rhodium, iridium, nickel, palladium and platinum. Besides their catalytic activity, suitable catalysts may be inert under the selected reaction conditions.

The metals may be present in any chemical form, for example in elemental, colloidal, salt or oxide form, in combination with complexing agents such as chelates, or as alloys, in which case the alloys may also include other metals, for example aluminium, as well as the metals listed above.

The metals may be present in supported form, i.e. applied to any support, preferably an inorganic support. Examples of suitable supports are carbon (charcoal or activated carbon), aluminium oxide, silicon dioxide, zirconium dioxide or titanium dioxide. Catalysts preferred in accordance with the invention are nickel and cobalt as sponge metals (Raney-type catalyst) or as nanoparticles, more preferably cobalt as sponge metal.

In the catalytic hydrogenation according to the invention, the catalyst is used in an amount of about 0.01 to about 30% by weight based on compound of formula (VIII). The catalyst is preferably used in a concentration of about 10 to about 30% by weight. Or compound (VIII) can be added as solid or as solution to an excess of catalyst in a solvent in hydrogen gas atmosphere.

The catalytic hydrogenation can be performed under elevated pressure (i.e. up to about 200 bar) in an autoclave, or at standard pressure in a hydrogen gas atmosphere. Especially at high reaction temperatures, it may be helpful to work at elevated pressure. The (additional) pressure increase can be brought about by supply of an inert gas, such as nitrogen or argon. The inventive catalytic hydrogenation is effected preferably at a hydrogen gas pressure in the range from about 1 to about 50 bar, more preferably at a hydrogen gas pressure in the range from 10 to 30 bar.

It is generally advantageous to perform the catalytic hydrogenation in the presence of solvents (diluents). Solvents are advantageously used in such an amount that the reaction mixture can efficiently be stirred over the entire process. Advantageously, based on compound (VIII) used, 1 to 50 times the amount of solvent, preferably 1 to 10 times the amount of solvent and more preferably 2 to 5 times the amount of solvent is used.

Useful solvents for performance of catalytic hydrogenation according to the present invention include all organic solvents which are inert under the reaction conditions. The solvent used depends on the type of reaction procedure, more particularly on the type of catalyst used and/or the hydrogen source (introduction of gaseous hydrogen or generation in situ). Mixtures of solvents can also be used.

Solvents suitable for the catalytic hydrogenation are alcohols such as methanol, ethanol, isopropanol, butanol; ethers, such as ethyl propyl ether, methyl tert-butyl ether, n-butyl ether, anisole, phenetole, cyclohexyl methyl ether, dimethyl ether, diethyl ether, dimethylglycol, diphenyl ether, dipropyl ether, diisopropyl ether, di-n-butyl ether, diisobutyl ether, diisoamyl ether, ethylene glycol dimethyl ether, isopropyl ethyl ether, methyl tert-butyl ether, tetrahydrofuran, dioxane, dichlorodiethyl ether, and polyethers of ethylene oxide and/or propylene oxide; aliphatic, cycloaliphatic or aromatic hydrocarbons such as pentane, hexane, heptane, octane, nonane, and technical-grade hydrocarbons which may be substituted by fluorine and chlorine atoms, such as methylene chloride, dichloromethane, trichloromethane, carbon tetrachloride, fluorobenzene, chlorobenzene or dichlorobenzene; for example white spirits having components with boiling points in the range, for example, from 40° C. to 250° C., cymene, petroleum fractions within a boiling range from 70° C. to 190° C., cyclohexane, methylcyclohexane, petroleum ether, ligroin, octane, benzene, toluene, chlorobenzene, bromobenzene, xylene; esters such as methyl acetate, ethyl acetate, propyl acetate, isopropyl acetate, butyl acetate, isobutyl acetate, and also dimethyl carbonate, dibutyl carbonate or ethylene carbonate. Another solvent is water.

The catalytic hydrogenation can optionally be performed in the presence of acids or bases. Acids suitable for the catalytic hydrogenation are inorganic acids, such as hydrochloric acid, hydrobromic acid, sulphuric acid, phosphoric acid; organic acids, such as acitic acid, trichloro acetic acid, trifluoro acetic acid and benzoic acid. Bases suitable for the catalytic hydrogenation are inorganic bases, e.g. alkali metal carbonates, such as sodium carbonate, potassium carbonate; alkaline earth metal carbonates, such as calcium carbonate; organic bases, e.g. alkylamines, such as triethylamine and ethyl di-iso-propyl amine.

In the catalytic hydrogenation according to the invention, the solvents used are preferably esters, ethers or alcohols without acids or bases.

The catalytic hydrogenation according can be performed within a wide temperature range (for example in the range from about −20° C. to about 120° C.). Preference is given to performing the catalytic hydrogenation within a temperature range from about 20° C. to about 100° C., in particular room temperature (i.e. around 20° C.) to 80° C.

The catalytic hydrogenation can be performed in a batch process where compound of formula (VIII) and the catalyst are added to a reaction vessel preferably in the presence of a solvent at once and then reacted under suitable temperature and hydrogen pressure. Alternatively, the reaction can also be performed as a semi-batch process, where the compound of formula (VIII) is added controlled as a solid or as a solution in a suitable solvent over a period of time to the reaction vessel containing a mixture of catalyst in solvent under suitable temperature and hydrogen pressure. Compound of formula (VIII) can be added over a time period of 30 min to 24 h, preferably 1-8 h.

Furthermore, the present invention relates to an intermediate compound in route (B), where a compound of the general formula (I) is produced by a Heck reaction and in-situ cyclisation of a compound of the general formula (III) with an acrylate compound of the general formula (IV). The intermediate compound of the general formula (V)

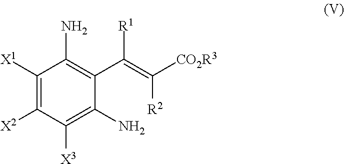

in which X¹, X², X³, R¹ and R² are as defined in formula (I), and R³ is as defined in formula (IV) can be isolated between the Heck reaction and the cyclisation reaction by appropriate measures of the reaction.

Route C

Step (C1)

The process for the preparation of a 5-amino-quinolin-2(1H)-one compound according to route (C) is a cyclisation of a compound of the genera formula (V)

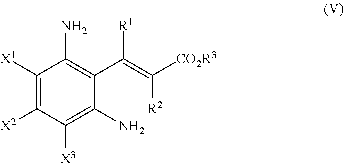

in which X¹, X², X³, R¹ and R² are as defined in formula (I), and R³ is as defined in formula (IV) in presence of an activation agent.

The activation agent for the cyclisation reaction of the compound of the general formula (V) can be selected from the group consisting of acids and bases, preferably bases.

Examples of bases which may be used in step (C1) include alkali metal bases (e.g. sodium carbonate, potassium carbonate, lithium hydride, sodium hydride, potassium hydride, butyl lithium, tert-butyl lithium, trimethylsilyl lithium, lithium hexamethyldisilazide, cesium carbonate, tripotassium phosphate, sodium acetate, potassium acetate, sodium methoxide, sodium ethoxide, sodium-tert-butoxide and potassium-tert-butoxide) or organic bases (e.g. triethylamine, diisopropylethylamine, tributylamine, N-methylmorpholine, N,N-dimethylaniline, N,N-diethylaniline, 4-tert-butyl-N,N-dimethylaniline, 4-tert-butyl-N,N-diethylaniline, pyridine, picoline, lutidine, diazabicyclooctan (DABCO), diazabicyclononen (DBN), bicycloundecen (DBU) and imidazole). Preferred bases are sodium carbonate, potassium carbonate and sodium methoxide.

Suitable acids include inorganic acids (e.g. hydrochloric acid, hydrobromic acid, sulfuric acid, phosphoric acid, poly phosphoric acid, nitric acid), organic acids (acetic acid, trifluoro acetic acid, methane sulfonic acid, para-toluene sulfonic acid, camphorsulfonic acid). Preferred acids are acetic acid and hydrochloric acid.

The molar ratio of the activation agent used to the compound of the general formula (V) (activation agent: compound of formula (V)) can vary. Preferably the molar ratio is in the range of 1:1 to 5:1.

The cyclisation reaction of route (C) according to the present invention can be carried out in an organic solvent. The solvent is preferably used in an amount such that the reaction mixture is readily stirrable during the entire process. The solvent is preferably inert under the reaction conditions. According to the present invention, solvents are also understood as meaning mixtures of solvents, preferably pure solvents.

The solvents suitable for this reaction can be selected from the group consisting of alcohols (e.g. methanol, ethanol, propanol, iso-propanol, butanol), aprotic solvents (e.g. acetonitrile, propionitrile, N,N-dimethylformamide, N,N-dimethyl acetamide), and water and mixtures of the aforementioned solvents.

The cyclisation reaction of route (C) according to the present invention can generally be carried out in vacuum, at atmospheric pressure or under superatmospheric pressure. Preferably the reaction is carried out at atmospheric pressure.

The temperatures can vary depending on the specific compound of the general formula (V) used for the cyclisation reaction. The cyclisation reaction of route (C) according to the invention can be carried out at temperatures in the range of 0° C. to 160° C.

Step (C2)

The compound of the general formula (V) can be produced by the reduction of both nitro groups of a compound of the general formula (IX)

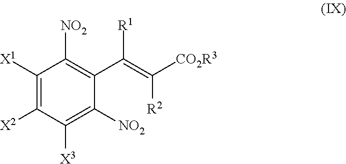

in which X¹, X², X³, R¹ and R² are as defined in formula (I), and R³ is as defined in formula (IV).

The reduction of both nitro groups of the compound of the general formula (IX) in step (C2) is preferably selected from a metal reduction under acidic conditions, a reduction with sulfides or a catalytic hydrogenation.

The metal reduction under acidic is preferably carried out in the presence of at least one reducing agent of the group consisting of a metal, preferably selected from Fe, Sn or Zn, in combination with an acid, preferably acetic acid (HOAc), trifluoro acetic acid, hydrochloric acid (HCl), phosphoric acid or sulfuric acid, or combinations of metals, preferably selected from Fe or Zn in combination with salts selected from ammonium chloride, calcium chloride, iron (III) chloride. The reduction with sulfides is preferably carried out by using a sulfide, preferably H₂S together with a base (e.g. NaOH, NH₄OH), Na₂S, NaHS, Na₂S₂, (NH₄)₂S, NH₄HS, (NH₄)₂S₂ or mixtures of the aforementioned salts with sulfur (generating polysulfides), or other inorganic reducing agents, preferably sodium dithionite (Na₂S₂O₄), sodium bisulfite (NaHSO₃) or tin(II)chloride.

The amount of the reducing agent in the metal reduction used to the compound of formula (IX) (reducing agent: compound of formula (IX)) can vary. Preferably the molar ratio is in the range 1:1 to 10:1, preferably 1:1 to 5:1.

The amount of the reducing agent in the reduction with sulfides used to the compound of formula (IX) (reducing agent: compound of formula (IX)) can vary. Preferably the molar ratio is in the range 1:1 to 10:1, preferably 1:1 to 5:1.

The reduction of step (C2) of the compound of the general formula (IX) according to the present invention can be carried out in an organic solvent. The solvent is preferably used in an amount such that the reaction mixture is readily stirrable during the entire process. The solvent is preferably inert under the reaction conditions. According to the invention, solvents are also understood as meaning mixtures of solvents, preferably pure solvents.

The organic solvents suitable for this reaction can be selected from the group consisting of alcohols (e.g. methanol, ethanol, isopropanol, butanol), ethers (e.g. methyl tert-butyl ether, n-butyl ether, anisole, tetrahydrofuran, dioxane, and polyethers of ethylene oxide and/or propylene oxide), aliphatic, cycloaliphatic or aromatic hydrocarbons (e.g. pentane, hexane, heptane, octane, nonane, and technical-grade hydrocarbons which may be substituted), esters (e.g. methyl acetate, ethyl acetate, butyl acetate, isobutyl acetate, and also dimethyl carbonate, dibutyl carbonate or ethylene carbonate), polar aprotic solvents (e.g. N,N-dimethylformamid, N,N-dimethylacetamid, acetonitrile, propionitril). Another solvent is water.

The reduction of step (C2) of a compound of the general formula (IX) according to the invention can generally be carried out in vacuum, at atmospheric pressure or under superatmospheric pressure. Preferably the reaction carried out at atmospheric pressure.

The temperatures applied during this process step can vary depending on the specific compound of formula (IX) used for the reduction. The reduction in route (C) of a compound of the general formula (IX) according to the present invention can be carried out at temperatures in the range of 0° C. to 150° C. Preferably the reaction is carried ouzt at temperatures in the range of 20° C. to 100° C.

The reduction of the nitro group to an amino group can be also carried out by catalytic hydrogenation. Suitable catalysts to be used for the catalytic hydrogenation in step (C2) of route (C) comprise one or more metals of groups 8 to 10 of the Periodic Table, especially one or more metals selected from iron, ruthenium, osmium, cobalt, rhodium, iridium, nickel, palladium and platinum. Besides their catalytic activity, suitable catalysts may be inert under the selected reaction conditions.

The metals may be present in any chemical form, for example in elemental, colloidal, salt or oxide form, in combination with complexing agents such as chelates, or as alloys, in which case the alloys may also include other metals, for example aluminium, as well as the metals listed above.

The metals may be present in supported form, i.e. applied to any support, preferably an inorganic support. Examples of suitable supports are carbon (charcoal or activated carbon), aluminium oxide, silicon dioxide, zirconium dioxide or titanium dioxide. Catalysts preferred in accordance with the invention contain one or more metals of groups 8 to 10 of the Periodic Table on an inorganic support. Particular preference is given in accordance with the invention to catalysts which include palladium and platinum, and are optionally applied to an inorganic support (e.g. carbon). Such catalysts are, for example, platinum on carbon, platinum oxide on carbon and palladium on carbon.

In the catalytic hydrogenation according to the invention, the catalyst is used in an amount of about 0.01 to about 30% by weight based on compound of formula (IX). The catalyst is preferably used in a concentration of about 0.1 to about 15% by weight.

The catalytic hydrogenation can be performed under elevated pressure (i.e. up to about 200 bar) in an autoclave, or at standard pressure in a hydrogen gas atmosphere. Especially at high reaction temperatures, it may be helpful to work at elevated pressure. The (additional) pressure increase can be brought about by supply of an inert gas, such as nitrogen or argon. The inventive catalytic hydrogenation is effected preferably at a pressure in the range from about 1 to about 30 bar, more preferably at a pressure in the range from about 5 to about 25 bar.

It is generally advantageous to perform the catalytic hydrogenation in the presence of solvents (diluents). However, the catalytic hydrogenation can also be performed without a solvent. Solvents are advantageously used in such an amount that the reaction mixture can efficiently be stirred over the entire process. Advantageously, based on compound (IX) used, 1 to 50 times the amount of solvent, preferably 2 to 40 times the amount of solvent and more preferably 2 to 30 times the amount of solvent is used.

Useful solvents for performance of catalytic hydrogenation according to the present invention include all organic solvents which are inert under the reaction conditions. The solvent used depends on the type of reaction procedure, more particularly on the type of catalyst used and/or the hydrogen source (introduction of gaseous hydrogen or generation in situ). Mixtures of solvents can also be used.

Solvents suitable for the catalytic hydrogenation are halohydrocarbons, e.g. chlorohydrocarbons, such as tetrachloroethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene; alcohols such as methanol, ethanol, isopropanol, butanol; ethers, such as ethyl propyl ether, methyl tert-butyl ether, n-butyl ether, anisole, phenetole, cyclohexyl methyl ether, dimethyl ether, diethyl ether, dimethylglycol, diphenyl ether, dipropyl ether, diisopropyl ether, di-n-butyl ether, diisobutyl ether, diisoamyl ether, ethylene glycol dimethyl ether, isopropyl ethyl ether, methyl tert-butyl ether, tetrahydrofuran, dioxane, dichlorodiethyl ether, and polyethers of ethylene oxide and/or propylene oxide; aliphatic, cycloaliphatic or aromatic hydrocarbons such as pentane, hexane, heptane, octane, nonane, and technical-grade hydrocarbons which may be substituted by fluorine and chlorine atoms, such as methylene chloride, dichloromethane, trichloromethane, carbon tetrachloride, fluorobenzene, chlorobenzene or dichlorobenzene; for example white spirits having components with boiling points in the range, for example, from 40° C. to 250° C., cymene, petroleum fractions within a boiling range from 70° C. to 190° C., cyclohexane, methylcyclohexane, petroleum ether, ligroin, octane, benzene, toluene, chlorobenzene, bromobenzene, xylene; esters such as methyl acetate, ethyl acetate, butyl acetate, isobutyl acetate, and also dimethyl carbonate, dibutyl carbonate or ethylene carbonate. Another solvent is water.

The catalytic hydrogenation can optionally be performed in the presence of acids or bases. Acids suitable for the catalytic hydrogenation are inorganic acids, such as hydrochloric acid, hydrobromic acid, sulphuric acid, phosphoric acid; organic acids, such as acitic acid, trichloro acetic acid, trifluoro acetic acid and benzoic acid. Bases suitable for the catalytic hydrogenation are inorganic bases, e.g. alkali metal carbonates, such as sodium carbonate, potassium carbonate; alkaline earth metal carbonates, such as calcium carbonate; organic bases, e.g. alkylamines, such as triethylamine and ethyl di-iso-propyl amine.

In the catalytic hydrogenation according to the invention, the solvents used are preferably ethers or alcohols.

The catalytic hydrogenation according can be performed within a wide temperature range (for example in the range from about −20° C. to about 100° C.). Preference is given to performing the catalytic hydrogenation within a temperature range from about 0° C. to about 100° C., in particular room temperature (i.e. around 20° C.).

Step (C3)

The compound of the general formula (IX) can be produced by a Heck reaction of a compound of the general formula (VIII)

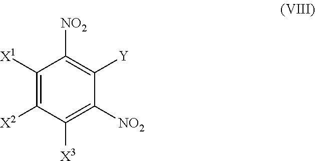

in which X¹, X² and X³ are as defined in formula (I), and Y is selected from Cl or Br, with the proviso that Y═Cl or Br when X¹, X² and X³═F, H or Y═Br when one of X¹, X² or X³═C1; with an acrylate compound of the general formula (IV)

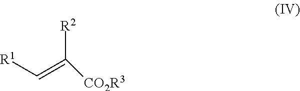

in which R¹ and R² are as defined in formula (I); and R³ is selected from the group consisting of hydrogen, substituted or unsubstituted C₁-C₁₂-alkyl, substituted or unsubstituted C₂-C₁₂-alkenyl, substituted or unsubstituted C₃-C₈-cycloalkyl, substituted or unsubstituted C₁-C₁₂-alkoxy, C₁-C₁₂-alkylaryl and aryl, or any salt thereof.

The acrylate compound within the Heck reaction in step (C3) according to the invention can be selected from the group consisting of acrylic acid and its salts (e.g. sodium acylate, potassium acrylate), methyl acrylate, ethyl acrylate, propyl acrylate, iso-propyl acrylate, butyl acrylate, phenyl acrylate, benzyl acrylate.

The stoichiometry of the compounds of the general formulae (VIII) and (IV) used can vary within wide ranges. The molar ratio of the compound of the general formula (VIII) to the acrylate compound of the general formula (IV) (compound of formula (VIII): compound of formula (IV)) used can be in the range 1:1 to 1:10.

A Heck reaction is a C—C coupling reaction with the use of a catalyst. The Heck reaction of route (C) according to the present invention can be catalyzed by different palladium catalysts.

A palladium catalyst contains a palladium source and a ligand. The palladium catalyst can be formed in situ or be added as a preformed catalyst system. It is also possible to perform Heck reaction using a palladium source and e.g. the base as ligand (known as phosphine free Heck reaction).

The palladium source can be selected from the group consisting of palladium(II)acetate, palladium(II) chloride, palladium(II) bromide, tris(dibenzylidene-acetone)dipalladium(0), Tris(dibenzylideneacetone)dipalladium-chloroform adduct, bis(dibenzylideneacetone)palladium(0), bis(acetonitrile)palladium(II) chloride, and allylpalladium chloride dimer.

The ligand can be selected from the group consisting of aryl and heteroaryl phosphines (e.g. triphenylphosphine, tri-ortho-tolylphosphine, trimesitylphosphine, tri-(2-furyl)phosphine, 2-(dicyclohexylphosphino)-2′-isopropylbiphenyl, 2-(Dicyclohexylphosphino)-2′,4′,6′-triisopropylbiphenyl, 2-(di-tert-butylphosphino) biphenyl), 2-(dicyclohexylphosphino)biphenyl, 2-dicyclohexylphosphino-2′-(N,Ndimethylamino) biphenyl, 2-diphenylphosphino-2′-(N,N-dimethylamino) biphenyl, 2-(dicyclohexylphosphino)-2′-methylbiphenyl, 2-(di-tert-butylphosphino)-2′-methylbiphenyl, 2-di-tert-butylphosphino-2′-(N,Ndimethylamino) biphenyl, 2-dicyclo-hexylphosphino-2′,6′-diisopropoxy-1,1′-biphenyl, 2-Di-tert-butylphosphino-2′,4′,6′-triisopropylbiphenyl), bidentate phosphines (e.g. (+)-2,2′-bis(diphenylphosphino)-1,1′-binaphthyl, bis(diphenylphosphino)methane, 1,2-bis(diphenylphosphino)ethane, 1,2-bis(dicyclohexylphosphino)ethane, 1,3-bis(diphenylphosphino)propane, 1,3-bis(dicyclohexylphosphino)propane, 1,4-bis(diphenylphosphino)butane, 1,5-bis(diphenylphosphino)pentane, bis(2-diphenylphosphinophenyl)ether, 1,1′-bis(di-phenylphosphino)ferrocene, 1,1′-bis(diisopropylphosphino)ferrocene, 1,1′-bis(di-tert-butylphosphino)ferrocene, 1,2-bis(diphenylphosphino)benzene), alkyl phosphines (e.g. tricyclohexylphosphine, triisopropylphosphine, tri-n-butylphosphine, di-tert-butylmethylphosphine, tri-tert-butylphosphine) and their salts (e.g. tri-tert-butylphosphine tetrafluorborate, tricyclohexylphosphine tetrafluorborate), phosphites (e.g. trimethyl phosphite, triethyl phosphite, tri-iso-propyl phosphite, tricyclohexyl phosphite, triphenyl phosphite, tri-2,4-tert-butyl phosphite), N-heterocyclic carbene precursors (e.g. 1,3-bis(2,4,6-trimethylphenyl)imidazolium chloride, 1,3-bis(2,6-diisopropylphenyl)imidazolium chloride, 1,3-bis(adamant-1-yl)imidazolium chloride, 1,3-bis(2,4,6-trimethylphenyl)-4,5-dihydroimidazolium tetrafluoroborate, 1,3-bis(2,6-diisopropylphenyl) imidazolidinium tetrafluoroborate, 1,3-bis(2,4,6-trimethylphenyl)imidazolidinium chloride and 1,3-bis(2,6-diisopropylphenyl)imidazolidinium chloride).

The preformed catalyst can be selected from the group consisting of palladium phosphine catalysts (e.g. bis(tri-tert-butylphosphine) palladium (0), [1,2-bis(diphenyl-phosphino)ethane]dichloropalladium(II), 1,1′-bis (diphenylphosphino)ferrocene-palladium(II)dichloride dichloromethane adduct, bis(tricyclohexylphosphine)-palladium(0), bis(triethylphosphine)palladium(II) chloride, bis(triphenylphosphine)-palladium(II) acetate, bis(triphenylphosphine)palladium(II) chloride, bis(tri-t-butylphosphine)palladium(0), bis[1,2-bis(diphenylphosphino)ethane]palladium(0), bis[tri(o-tolyl)phosphine]palladium(II)chloride, dichlorobis(tricyclohexyl-phosphine)palladium(II), tetrakis(triphenylphosphine)palladium(0) and trans-benzyl-(chloro)bis(triphenylphosphine)palladium(II)).

Additionally preformed catalysts might be prepared from the above palladium sources and ligands either in a separate reaction or in situ before the other reaction partners are added.

Usually, the Heck reaction of route (C) of the present invention can be carried out in the presence of an additive. Suitable additives include organic ammonium salts (e.g. tetrabutylammonium bromid, tetrabutylammonium chloride, tetrabuthylammonium bisulfate, tetrabutylammonium acetate, tetraethylammonium chlorid, tetraethylammonium bromid, benzyl trimethylammonium chlorid and benzyl trimethylammonium bromid), organic phosphonium salts (e.g. tetra-n-butylphosphoniumbromide) alkali and earth alkali salts (e.g. lithium chloride, lithium bromide, sodium chloride and magnesium chloride).

The Heck reaction of route (C) of the present invention can also be carried out in the presence of a base. Suitable bases include alkali metal salts (e.g. sodium carbonate, sodium bicarbonate, potassium carbonate, potassium bicarbonate, cesium carbonate, tripotassium phosphate, sodium acetate, potassium acetate, sodium methoxide, sodium ethoxide, sodium-tert-butoxide and potassium-tert-butoxide), organic bases (e.g. triethylamine, diisopropylethylamine, dicyclohexyl methylamine, tributylamine, N-methylmorpholine, N,N-dimethylaniline, N,N-diethylaniline, 4-tert-butyl-N,N-dimethylaniline, 4-tert-butyl-N,N-diethylaniline, pyridine, picoline, lutidine, and imidazole).

The molar ratio of the base or additive to the compound of formula (VIII) (base or additive: compound of formula (VIII)) can vary. Preferably the molar ratio is in the range of 1:1 to 2:1.

The Heck reaction of route (C) according to the present invention can be carried out in an aprotic organic solvent. The solvent is preferably used in an amount such that the reaction mixture is readily stirrable during the entire process. The solvent is preferably inert under the reaction conditions. According to the invention, solvents are also understood as meaning mixtures of solvents, preferably pure solvents.

The organic solvents suitable for this reaction can be selected from the group consisting of halohydrocarbons (e.g. chlorohydrocarbons, such as methylene chloride, 1,2-dichloroethane, chlorobenzene, bromobenzene, dichlorobenzene, chlorotoluene), alcohols (e.g. methanol, ethanol, isopropanol, butanol), ethers (e.g. methyl tert-butyl ether, n-butyl ether, anisole, tetrahydrofuran, dioxane, and polyethers of ethylene oxide and/or propylene oxide), aliphatic, cycloaliphatic or aromatic hydrocarbons (e.g. pentane, hexane, heptane, octane, nonane, and technical-grade hydrocarbons which may be substituted), esters (e.g. methyl acetate, ethyl acetate, butyl acetate, isobutyl acetate, and also dimethyl carbonate, dibutyl carbonate or ethylene carbonate) and polar aprotic solvents (e.g. N,N-dimethylformamid, N,N-dimethylacetamid, acetonitrile, propionitril). Another solvent is water.

The Heck reaction of route (C) according to the present invention can generally be carried out in vacuum, at atmospheric pressure or under superatmospheric pressure. Preferably the reaction is carried out at atmospheric pressure.

The temperatures can vary depending on the specific compounds of formulae (VIII) and (IV) used for the Heck reaction. The Heck reaction of route (C) according to the present invention can be carried out at temperatures in the range of 20° C. to 160° C.

Another aspect of the present invention is a compound of the following formula (X)

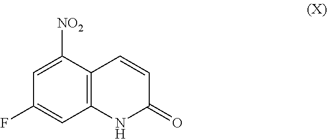 [SMILES: O=c1ccc2c([N+](=O)[O-])cc(F)cc2[nH]1]

Furthermore, the present invention also comprises a compound of the following formula (XI)

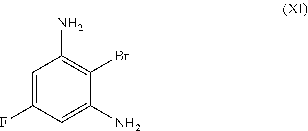

The present invention also relates to the compounds of the following formulae (XIIa and XIIb)

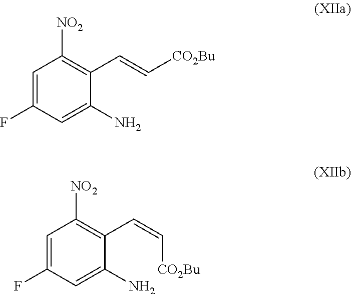

Additionally, a compound of the following formula (XIII) is also part of the present invention

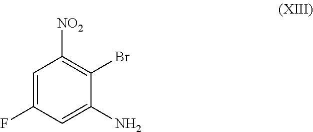 [SMILES: Nc1cc(F)cc([N+](=O)[O-])c1Br]

Finally, the present invention also comprises a compound of the following formula (XIV)

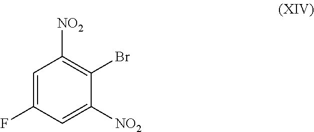 [SMILES: O=[N+]([O-])c1cc(F)cc([N+](=O)[O-])c1Br]

If not defined otherwise herein, and within the context of the present invention, the term “alkyl”, either alone or in combination with further terms such as, for example haloalkyl and arylalkyl, is understood as meaning a radical of a saturated, aliphatic hydrocarbon group having 1 to 12 carbon atoms, which may be branched or unbranched. Examples of C₁-C₁₂-alkyl radicals are methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1-ethylpropyl, 1,2-dimethylpropyl, hexyl n-heptyl, n-octyl, n-nonyl, n-decyl, n-undecyl and n-dodecyl.

If not defined otherwise herein, and within the context of the present invention, the terms “halogen atoms” or “halo”, either alone or in combination with further terms such as, for example haloalkyl, are understood as meaning radicals mono- or polyhalogenated up to the maximum possible number of substituents. In the case of polyhalogenation, the halogen atoms may be identical or different. Here, halogen is fluorine, chlorine, bromine or iodine.

If not defined otherwise herein, and within the context of the present invention, the term “alkenyl” is understood as meaning a linear or branched C₂-C₁₂-alkenyl radical which has at least one double bond, for example vinyl, allyl, 1-propenyl, isopropenyl, 1-butenyl, 2-butenyl, 3-butenyl, 1,3-butanedienyl, 1-pentenyl, 2-pentenyl, 3-pentenyl, 4-pentenyl, 1,3-pentanedienyl, 1-hexenyl, 2-hexenyl, 3-hexenyl, 4-hexenyl, 5-hexenyl and 1,4-hexanedienyl.

If not defined otherwise herein, and within the context of the present invention, the term “cycloalkyl” is understood as meaning a C3-C₈-cycloalkyl radical, for example cyclopropyl, cyclobutyl, cyclopentyl, cyclohexyl, cycloheptyl and cyclooctyl.

If not defined otherwise herein, and within the context of the present invention, the term “alkoxy” is understood in the present case as meaning an O-alkyl radical.

If not defined otherwise herein, and within the context of the present invention, the term “arylalkyl” is understood as meaning a combination of “aryl” and “alkyl” radicals defined according to the invention, the radical generally being bonded via the alkyl group. Examples thereof are benzyl, phenylethyl or a-methylbenzyl, with benzyl being particularly preferred.

If not defined otherwise herein, and within the context of the present invention, the term “aryl” is understood as meaning an aromatic radical having 6 to 14 carbon atoms, preferably phenyl.

If not defined otherwise herein, and within the context of the present invention, the term “substituted” in combination with further terms such as, for example substituted alkyl or substituted alkenyl is understood as meaning that a radical is substituted with one or more substituents. Examples for substituents are C₁-C₁₂-alkyl, C₂-C₁₂-alkenyl, C₃-C₈-cycloalkyl, C₁-C₁₂-alkoxy, alkylaryl and aryl, having the aforementioned meaning. The substituted radical may be mono- or polysubstituted, where, in the case of a poly-substitution, the substituents may be identical or different. The term “unsubstituted” indicates that the molecule is not substituted with one or more groups of atoms, but only with hydrogen atoms.

### EXAMPLES

The invention is illustrated by the following examples without limiting the invention to the same.

The following reactions for the subsequent preparation of a compound according to formula (I) via different steps and different intermediate product compounds also include the preparation of every tautomer of the compounds (e.g. lactam-lactim-tautomerism).

The following abbreviations are used:

MW=molecular weight, MS=mass spectrometry, GC=gas chromatography, NMR=nuclear magnetic resonance.

The analytical data given below have been collected using the following instruments:

NMR: Bruker Avance III (400 MHz) or BRUKER Avance III (600 MHz), measured at 300K;

LC-MS: Waters Acquity UPLC with 3100 Mass Detector.

GC: Perkin Elmer Autosystem XL, column: HP5, carrier gas: helium

GC-MS: Agilent 6890 GC, column: DB-1, 10 m, iD 0.18 mm, film 0.4 μm, injector: 250° C., flow: 1.6 mm/min He, oven: 0 min 50° C., 1 min 50° C., 7.75 min 320° C., 11 min 320° C., Hewlett-Packard 5973 MSD, MSD: 280° C., EI

### Preparation of Starting Material

### Preparation of 2-bromo-5-fluoro-1,3-dinitrobenzene

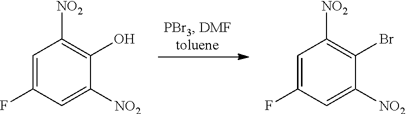 [SMILES: O=[N+]([O-])c1cc(F)cc([N+](=O)[O-])c1O ; O=[N+]([O-])c1cc(F)cc([N+](=O)[O-])c1Br]

4-Fluoro-2,6-dinitrophenol (CAS 364-32-9, 16.1 g, 79.4 mmol as a 32 weight % solution in toluene) was placed in a reaction vessel and DMF (24.4 mL, 317 mmol) was added. The reaction mixture was heated to 80° C. and then phosphorus tribromide (11.7 mL, 119 mmol) was added over 20 minutes. The reaction was stirred for another four hours at 77° C. and after cooling was left standing over night. The reaction was again heated to 85° C. for three hours and at that point all starting material had reacted. After cooling to 60° C. water (100 mL) was added to the reaction mixture (exotherm to 80° C.). The phases were separated and the aqueous phase was extracted with toluene (50 mL). The combined organic phases were successively washed with water (2×20 mL), saturated sodium chloride solution (10 mL) and dried over magnesium sulfate. After filtration the filtrate was concentrated under vacuum. n-Heptane was added and the resulting suspension was concentrated even further. 2-Bromo-5-fluoro-1,3-dinitrobenzene (17.1 g, purity 99 area % HPLC, yield 80%) was isolated through filtration.

¹H-NMR (CDCl₃, 400 MHz): δ=7.72 ppm (d, ³J=8.0 Hz, 2H); ¹³C-NMR (CDCl₃, 150.9 MHz): δ=160.6 (¹J_{C-F}=259 Hz), 152.2 (br.), 116.0 (²J_{C-F}=26.6 Hz), 102.8 ppm (⁴J_{C-F}=5.2 Hz); LC-MS (ESI−): m/z=201.0 [(M−1)⁻].

### Preparation Example 1

### Preparation of 2-bromo-5-fluorobenzene-1,3-diamine (step (B2))

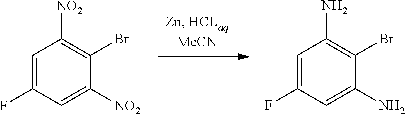 [SMILES: O=[N+]([O-])c1cc(F)cc([N+](=O)[O-])c1Br ; Nc1cc(F)cc(N)c1Br]

2-Bromo-5-fluoro-1,3-dinitrobenzene (41.0 g, 153 mmol) and zinc (66.4 g, 995 mmol) were put in a reaction vessel and suspended in acetonitrile (267 mL). Hydrochloric acid (176 mL, 35% in water, 1.99 mol) was slowly added at 20-30° C. (exotherm! Temperature held with ice water cooling) over one hour. After one hour more zinc (1.3 g, 19.9 mmol) was added and after an additional hour the starting material and the reaction intermediates were converted to the product. The acidic reaction mixture was treated with saturated sodium bicarbonate solution (95.0 g, 8.6% in water, 97.3 mmol). Acetonitrile was removed under vacuum at 40° C. yielding a darkened suspension of the product. The product was extracted with ethyl acetate and the organic phase was washed with saturated sodium chloride solution, dried over magnesium sulfate and the drying agent was filtered off. After standing overnight a dark solid appeared which was dissolved in ethyl acetate. Activated charcoal was added and after stirring for a while filtered off. To the filtrate n-heptane was added and the mixture was concentrated on the rotatory evaporator to a thick suspension. The solid was filtered off and washed with n-heptane to give 2-bromo-5-fluorobenzene-1,3-diamine (27.4 g, purity 99 area % HPLC, yield 86%) as off white solid.

¹H-NMR (CDCl₃, 400 MHz): δ=5.94 (d, ³J=8.0 Hz, 2H), 4.13 ppm (br. S, 4H); ¹³C-NMR (CDCl₃, 150.9 MHz): δ=163.3 (¹J_{C-F}=240 Hz), 145.7 (³J_{C-F}=13.5 Hz), 92.4 (²J_{C-F}=26.6 Hz), 91.5 ppm (⁴J_{C-F}=2.5 Hz); LC-MS (ESI+): m/z=205.0; 207.0 [(M+1)⁺].

### Alternative Preparation of 2-bromo-5-fluorobenzene-1,3-diamine (step (B2))

A mixture of 2-Bromo-5-fluoro-1,3-dinitrobenzene (5.0 g, 18.87 mmol), ethyl acetate (25 g, 27.8 ml) and sponge cobalt catalyst (1.15 g, Raney-type cobalt, BASF: Actimet Co, previously washed with ethanol (2 times) and ethyl acetate (2 times)) was stirred in a Parr autoclave at 60° C. under 20 bar hydrogen pressure for 3 hours. Then the autoclave was cooled to 20° C. and the pressure was released to atmospheric pressure. The suspension was filtered into a flask containing 100 ml heptane and the filter was washed with 5 ml ethyl acetate. The filtrate was concentrated on a rotatory evaporator to obtain 2-bromo-5-fluorobenzene-1,3-diamine as a beige shiny solid (3.7 g, 96% purity by ¹H-NMR, yield 92%).

GC-MS (EI⁺): Rₜ=4.445 min (index 1488, 100 area %), m/z=204/206 [M⁺].

¹H-NMR (CDCl₃, 400 MHz): δ=4.14 (br, 4H), 5.94 (d, ³J=10.4 Hz, 2H) ppm.

### Preparation of 5-amino-7-fluoroquinolin-2(1H)-one (or its tautomer 5-amino-7-fluoroquinolin-2-ol) (step (B1))

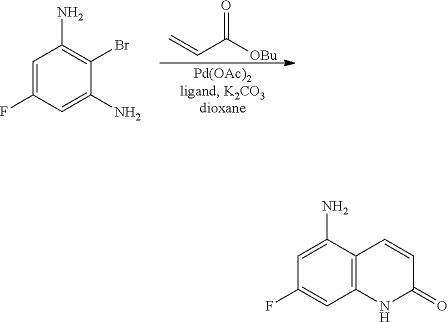

2-Bromo-5-fluorobenzene-1,3-diamine (10.0 g, 48.3 mmol) was dissolved in 1,4-dioxane (65.0 mL) and nitrogen was bubbled through the solution for 15 minutes to degas the solution. Palladium (II) acetate (152 mg, 676 μmol), 2-Dicyclohexylphosphino-2′,4′,6′-triisopropylbiphenyl (365 mg, 744 μmol) and potassium carbonate (8.01 g, 57.9 mmol) were added. After the addition of butyl acrylate (7.56 mL, 53.1 mmol) the reaction mixture was heated to reflux for 5 hours and 50 minutes. After standing over night at room temperature the reaction was heated to reflux for another four hours and 30 minutes. The formed solid was isolated by filtration; the filter cake was washed with ethyl acetate, then suspended in water (60 mL) and filtration yielded 5-amino-7-fluoroquinolin-2(1H)-one (7.15 g, purity >98 area % HPLC, yield 81%) as a beige solid.

¹H-NMR (DMSO-D⁶, 400 MHz): δ=11.48 (br. s, 1H), 8.05 (d, ³J=9.8 Hz, 1H), 6.24 (s, 2H), 6.22 (d, ³J=9.9 Hz, 1H), 6.19 (dd, ³J_{H-F}=10.6 Hz, ⁴J=2.6 Hz, 1H), 6.13 ppm (dd, ³J_{H-F}=11.9 Hz, 4J=2.5 Hz, 1H); ¹³C-NMR (DMSO-D⁶, 150.9 MHz): δ=164.3 (¹J_{C-F}=241 Hz), 162.2, 148.3 (³J_{C-F}=15.0 Hz), 141.7 (³J_{C-F}=15.0 Hz), 135.2, 116.6, 103.1, 92.9 (²J_{C-F}=25.7 Hz), 88.2 ppm (d, ²J_{C-F}=34.7 Hz); LC-MS (ESI+): m/z=179.0 [(M⁺1)⁺].

### Preparation Example 2

### Preparation of 2-bromo-5-fluoro-3-nitroaniline (step (A4))

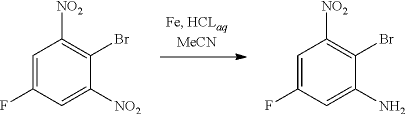 [SMILES: O=[N+]([O-])c1cc(F)cc([N+](=O)[O-])c1Br ; Nc1cc(F)cc([N+](=O)[O-])c1Br]

2-Bromo-5-fluoro-1,3-dinitrobenzene (11.6 g, 43.8 mmol) was dissolved in ethanol (50.0 mL) and iron powder (7.33 g, 131 mmol) were added. The mixture was heated to 60° C. and hydrochloric acid (35 w % aqueous solution, 23.2 mL, 263 mmol) were added over 45 minutes. The reaction was stirred at 80° C. for additional 75 minutes and then cooled to room temperature. Toluene (50 mL) and water were added and the mixture was filtered over a paper filter. The phases were separated and the aqueous phase was extracted with ethyl acetate (50 mL). The combined organic phases were washed with hydrochloric acid (5 w %, 50 mL) and then filtered over a plug of silica (eluent n-heptane/ethyl acetate 7/3). The solvents were evaporated and the product was obtained as a bright yellow solid (7.30 g, purity 88.9 area % HPLC, yield 72%).

¹H-NMR (DMSO-D⁶, 600 MHz): δ=7.07 (dd, ³J_{H-F}=8.2 Hz, ⁴J=2.9 Hz, 1H), 6.80 (dd, ³J_{H-F}=11.0 Hz, ⁴J=2.9 Hz, 1H), 6.33 ppm (br. s, 2H); ¹³C-NMR (DMSO-D⁶, 150.9 MHz): δ=161.1 (¹J_{C-F}=244.5 Hz), 151.7 (³J_{C-F}=12.1 Hz), 149.2 (³J_{C-F}=13.6 Hz), 103.0 (²J_{C-F}=25.7 Hz), 98.9 (²J_{C-F}=28.7 Hz), 92.3 ppm (⁴J_{C-F}=3.0 Hz); LC-MS (ESI+): m/z=235.0/237.0 [(M⁺1)⁺].

A use test in the following Heck reaction revealed that the iron content was too high for the reaction to work. Therefore the material was further purified by column chromatography (n-heptane/ethyl acetate on silica).

### Preparation of butyl (2E)-3-(2-amino-4-fluoro-6-nitrophenyl)acrylate (step A3 a)

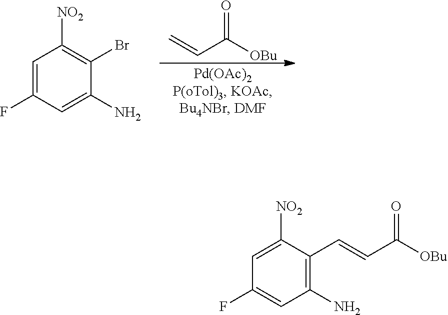

2-Bromo-5-fluoro-3-nitroaniline (2.00 g, purity 92 area % HPLC, 7.83 mmol) was dissolved in N,N-dimethyl formamide (15 mL) and the mixture was degassed by bubbling argon through the stirred reaction mixture for 45 minutes. Thereafter the following compounds were added in the following order: potassium acetate (1.92 g, 19.6 mmol), tetra-n-buthyl ammonium bromide (505 mg, 1.57 mmol), tris-ortho-tolyl phosphine (119 mg, 391 μmol) and buthyl acylate (2.00 g, 15.6 mmol). Then the reaction mixture was heated to 100° C. and one third of the palladium (II) acetate (in total 43.9 mg, 196 μmol) was added. After 15 minutes at 100° C. 4 area % HPLC of the starting material were left and another third of the palladium (II) acetate was added. Fifteen minutes later the last third of palladium (II) acetate was added and the reaction mixture was left cooling to 45° C. after another 15 minutes at 100° C. The warm reaction mixture was poured onto cold water (250 mL). After five minutes of stirring the mixture was extracted with ethyl acetate (50 mL) and the aqueous phase again was extracted with ethyl acetate (2×25 mL). The combined organic phases were dried over sodium sulfate and after evaporation of the solvents the product was obtained as a brown waxy solid (2.50 g, purity 73 area % HPLC, yield 83%).

¹H-NMR (DMSO-D⁶, 400 MHz): δ=7.49 (d, ³J=16.3 Hz, 1H), 7.02 (dd, ³J_{H-f}=8.4 Hz, ⁴J=2.6 Hz, 1H), 6.78 (dd, ³J_{H-f}=11.1 Hz, ⁴J=2.6 Hz, 1H), 6.23 (br. s, 2H), 6.13 (d, ³J=16.3 Hz, 1H), 4.14 (t, ³J=6.6 Hz, 2H), 1.66-1.57 (m, 2H), 1.42-1.32 (m, 2H), 0.91 ppm (t, ³J=7.4 Hz, 3H); ¹³C-NMR (DMSO-D⁶, 150.9 MHz): δ=165.5, 161.8 (¹J_{C-F}=245.8 Hz), 150.8 (³J_{C-F}=12.3 Hz), 150.1 (³J_{C-F}=12.6 Hz), 136.2, 123.1, 108.2 ppm (⁴J_{C-F}=2.5 Hz), 104.5 (²J_{C-F}=24.2 Hz), 98.9 (²J_{C-F}=28.5 Hz), 64.0, 30.2, 18.6, 13.6 ppm; LC-MS (ESI−): m/z=281.1 [(M−1)⁻]. The Z-Isomer was only obtained in minor amounts (less then 5%).

### Preparation of 7-fluoro-5-nitroquinolin-2(1H)-one (and its tautomer 7-fluoro-5-nitroquinolin-2-ol) (step (A2))

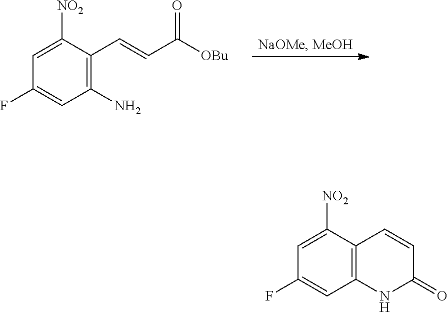

(2E)-3-(2-Amino-4-fluoro-6-nitrophenyl)acrylate (2.00 g, purity 80%, 5.67 mmol) was dissolved in methanol (40 mL) and sodium methylate (1.02 g, 30% solution in methanol) was added. The reaction was heated to 60° C., kept at that temperature for five hours minutes, then left standing over night at room temperature and then again heated to 60° C. for eight hours. The methanol was partially removed by distillation and ethyl acetate was added. The formed solid was filtered off and washed with more ethyl acetate. The thereby obtained sodium salt of the product was dissolved in water and the pH was adjusted to 2 with hydrochloric acid. The product was obtained by filtration as a brownish solid (730 mg, purity 97 area % HPLC, yield 60%). Additional product was found in the mother liquor of the first filtration. It was isolated after evaporation of the solvents through the procedure described above (210 mg, purity 68 area % HPLC, yield 12%).

¹H-NMR (DMSO-D⁶, 600 MHz): δ=12.30 (s, 1H), 8.18 (d, ³J=10.1 Hz, 1H), 7.87 (dd, ³J_{H-F}=8.7 Hz, ⁴J=2.5 Hz, 1H), 7.40 (dd, ³J_{H-F}=9.3 Hz, ⁴J=2.4 Hz, 1H), 6.72 ppm (d, ³J=10.1 Hz, 1H); ¹³C-NMR (DMSO-D⁶, 151 MHz): δ=160.8 (¹J_{C-F}=249.6 Hz), 160.8, 147.2 (³J_{C-F}=11.1 Hz), 141.5 (³J_{C-F}=12.3 Hz), 133.8, 124.3, 108.8 (⁴J_{C-F}=1.9 Hz), 107.3 (²J_{C-F}=28.8 Hz), 106.4 ppm (²J_{C-F}=25.0 Hz); LC-MS (ESI−): m/z=209.0 [(M−1)].

### Preparation of 5-amino-7-fluoroquinolin-2(1H)-one (step (A1))

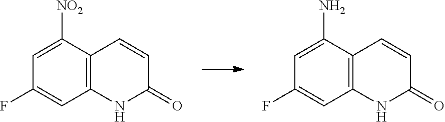 [SMILES: O=c1ccc2c([N+](=O)[O-])cc(F)cc2[nH]1 ; Nc1cc(F)cc2[nH]c(=O)ccc12]

7-fluoro-5-nitroquinolin-2(1H)-one (650 mg, purity 97 area %, 3.02 mmol) was dissolved in methanol (50.0 mL) and ammonium formate (1.52 g, 24.2 mmol) and palladium on charcoal (10% Pd, 50 mg, 47.0 μmol) were added. The reaction mixture was heated to 50° C. for 105 minutes, and then the catalyst was filtered off and washed with methanol. To the mother liquor water (20 mL) was added and the pH was adjusted to 5 with aqueous hydrochloric acid. A precipitate formed and after evaporation of some of the methanol even more precipitate formed. The solid was filtered off and washed with water. After drying the product was obtained as a light brown crystalline solid (504 mg, purity 98 area % HPLC, yield 91%).

¹H-NMR (DMSO-D⁶, 400 MHz): δ=11.48 (br. s, 1H), 8.05 (d, ³J=9.8 Hz, 1H), 6.24 (s, 2H), 6.22 (d, ³J=9.9 Hz, 1H), 6.19 (dd, ³J_{H-F}=10.6 Hz, ⁴J=2.6 Hz, 1H), 6.13 ppm (dd, ³J_{H-F}=11.9 Hz, 4J=2.5 Hz, 1H); ¹³C-NMR (DMSO-D⁶, 150.9 MHz): δ=164.3 (¹J_{C-F}=241 Hz), 162.2, 148.3 (³J_{C-F}=15.0 Hz), 141.7 (³J_{C-F}=15.0 Hz), 135.2, 116.6, 103.1, 92.9 (²J_{C-F}=25.7 Hz), 88.2 ppm (d, ²J_{C-F}=34.7 Hz); LC-MS (ESI+): m/z=179.0 [(M⁺1)⁺].

1. Process for preparation of a 5-amino-quinolin-2H(1H)-one compound of formula (I) 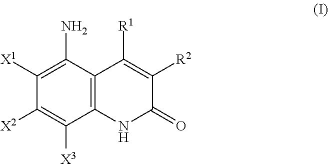
wherein
X¹, X² and X³ are identical or different and independently selected from the group consisting of H, F and C1; and
R¹ and R² may be same or different and are independently selected from the group consisting of H, C₁-C₁₂-alkyl, C₁-C₁₂-haloalkyl or halogen atoms;
wherein said compound is either produced by
route (A), reduction of the nitro-group of a compound of formula (II) 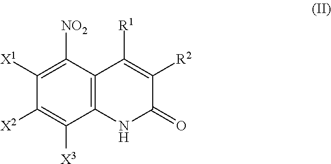
wherein
X¹, X², X³, R¹ and R² are as defined in formula (I)
or by
route (B), Heck reaction and in-situ cyclisation of a compound of formula (III), 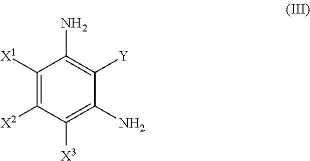
wherein
X¹, X² and X³ are as defined in formula (I), and
Y is selected from Cl or Br,
with the proviso that Y═Cl or Br when X¹, X² and X³═F, H or Y═Br when one of X¹, X² or X³═Cl
with an acrylate compound of formula (IV) 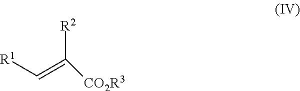
wherein
R¹ and R² are as defined in formula (I), and
R³ is selected from the group consisting of hydrogen, substituted or unsubstituted C₁-C₁₂-alkyl, substituted or unsubstituted C₂-C₁₂-alkenyl, substituted or unsubstituted C₃-C₈-cycloalkyl, substituted or unsubstituted C₁-C₁₂-alkoxy, C₁-C₁₂-alkylaryl and aryl,
or any salt thereof
or by
route (C), cyclisation of a compound of formula (V) 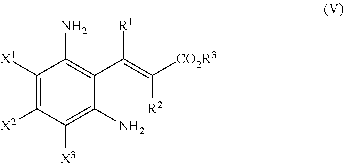
wherein
X¹, X² and X³ are identical or different and independently selected from the group consisting of H, F and Cl; and
R¹ and R² may be same or different and are independently selected from the group consisting of H, C₁-C₁₂-alkyl, C₁-C₁₂-haloalkyl or halogen atoms
and
R³ is as defined in formula (IV)
in presence of an activation agent.

2. The process according to claim 1, wherein in route (A) a compound of formula (II) is produced by cyclisation of a compound of formula (VI) 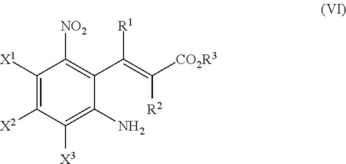
wherein
X¹, X² and X³ are identical or different and independently selected from the group consisting of H, F and Cl; and
R¹ and R² may be same or different and are independently selected from the group consisting of H, C₁-C₁₂-alkyl, C₁-C₁₂-haloalkyl or halogen atoms;
and
R³ is selected from the group consisting of hydrogen, substituted or unsubstituted C₁-C₁₂-alkyl, substituted or unsubstituted C₂-C₁₂-alkenyl, substituted or unsubstituted C₃-C₈-cycloalkyl, substituted or unsubstituted C₁-C₁₂-alkoxy, C₁-C₁₂-alkylaryl and aryl
in the presence of an activation agent.

3. The process according to claim 2, wherein in route (A), the compound of formula (VI) is produced by a Heck reaction of a compound of formula (VII) 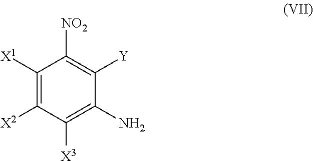
wherein
X¹, X² and X³ are identical or different and independently selected from the group consisting of H, F and Cl; and
R¹ and R² may be same or different and are independently selected from the group consisting of H, C₁-C₁₂-alkyl, C₁-C₁₂-haloalkyl or halogen atoms
and
Y is as defined in formula (III)
with an acrylate compound of formula (IV) 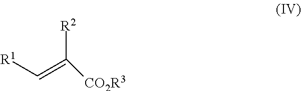
wherein
R¹ and R² may be same or different and are independently selected from the group consisting of H, C₁-C₁₂-alkyl, C₁-C₁₂-haloalkyl or halogen atoms; and
R³ is selected from the group consisting of hydrogen, substituted or unsubstituted C₁-C₁₂-alkyl, substituted or unsubstituted C₂-C₁₂-alkenyl, substituted or unsubstituted C₃-C₈-cycloalkyl, substituted or unsubstituted C₁-C₁₂-alkoxy, C₁-C₁₂-alkylaryl and aryl,
or any salt thereof
or by
reduction of one of the nitro-groups of a compound of formula (IX) 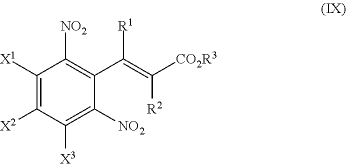
wherein
X¹, X² and X³ are identical or different and independently selected from the group consisting of H, F and C1; and
R¹ and R² may be same or different and are independently selected from the group consisting of H, C₁-C₁₂-alkyl, C₁-C₁₂-haloalkyl or halogen atoms
and
R³ is selected from the group consisting of hydrogen, substituted or unsubstituted C₁-C₁₂-alkyl, substituted or unsubstituted C₂-C₁₂-alkenyl, substituted or unsubstituted C₃-C₈-cycloalkyl, substituted or unsubstituted C₁-C₁₂-alkoxy, C₁-C₁₂-alkylaryl and aryl,

4. The process according to claim 3, wherein in route (A), the compound of formula (VII) is produced by a reduction of a compound of formula (VIII) 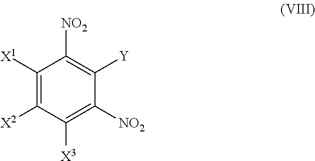
wherein
X¹, X² and X³ are identical or different and independently selected from the group consisting of H, F and Cl; and
R¹ and R² may be same or different and are independently selected from the group consisting of H, C₁-C₁₂-alkyl, C₁-C₁₂-haloalkyl or halogen atoms
and
X and Y are identical or different and are selected from the group of halogen atoms, with the proviso that Y═Cl, Br when X¹, X² and X³═F, H or Y═Br when one of X¹, X² or X³═Cl.

5. The process according to claim 1, wherein in route (B), a compound of formula (III) is produced by a reduction of both nitro groups of a compound of formula (VIII) 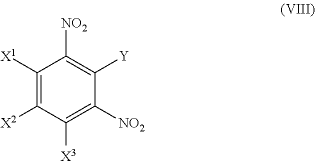
wherein
X¹, X² and X³ are identical or different and independently selected from the group consisting of H, F and Cl; and
R¹ and R² may be same or different and are independently selected from the group consisting of H, C₁-C₁₂-alkyl, C₁-C₁₂-haloalkyl or halogen atoms
and
X and Y are identical or different and are selected from the group of halogen atoms, with the proviso that Y═Cl, Br when X¹, X² and X³═F, H or Y═Br when one of X¹, X² or X³═Cl.

6. The process according to claim 1, wherein in route (C), a compound of formula (V) is produced by a reduction of both nitro groups of a compound of formula (IX) 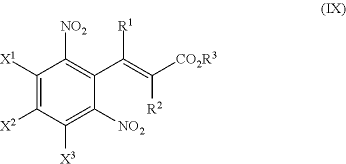
in which
X¹, X² and X³ are identical or different and independently selected from the group consisting of H, F and Cl; and
R¹ and R² may be same or different and are independently selected from the group consisting of H, C₁-C₁₂-alkyl, C₁-C₁₂-haloalkyl or halogen atoms
and
R³ is selected from the group consisting of hydrogen, substituted or unsubstituted C₁-C₁₂-alkyl, substituted or unsubstituted C₂-C₁₂-alkenyl, substituted or unsubstituted C₃-C₈-cycloalkyl, substituted or unsubstituted C₁-C₁₂-alkoxy, C₁-C₁₂-alkylaryl and aryl.

7. The process according to claim 6, wherein in route (C), the compound of formula (IX) is produced by a Heck reaction of a compound of formula (VIII) 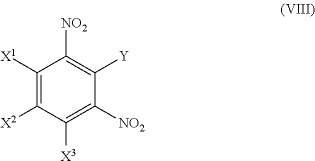
wherein
X¹, X² and X³ are identical or different and independently selected from the group consisting of H, F and Cl; and
R¹ and R² may be same or different and are independently selected from the group consisting of H, C₁-C₁₂-alkyl, C₁-C₁₂-haloalkyl or halogen atoms
and
X and Y are identical or different and are selected from the group of halogen atoms, with the proviso that Y═Cl, Br when X¹, X² and X³═F, H or Y═Br when one of X¹, X² or X³═Cl.
with an acrylate compound of the general formula (IV) 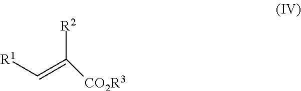
wherein
R¹ and R² are as above, and
R³ is selected from the group consisting of hydrogen, substituted or unsubstituted C₁-C₁₂-alkyl, substituted or unsubstituted C₂-C₁₂-alkenyl, substituted or unsubstituted C₃-C₈-cycloalkyl, substituted or unsubstituted C₁-C₁₂-alkoxy, C₁-C₁₂-alkylaryl and aryl,
or any salt thereof.

8. The process according to claim 1, wherein in route (B), a compound of formula (I) is produced by a Heck reaction and in-situ cyclisation of a compound of formula (III) with an acrylate compound of formula (IV), wherein an intermediate compound of formula (V) is obtained between the Heck reaction and the cyclisation reaction.

9. A compound of the following formula (X)

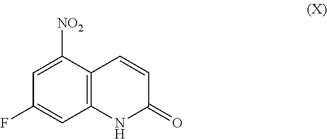 [SMILES: O=c1ccc2c([N+](=O)[O-])cc(F)cc2[nH]1]

10. A compound of the following formula (XI)

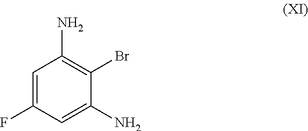

11. A compound of the following formulae (XIIa) and (XIIb)

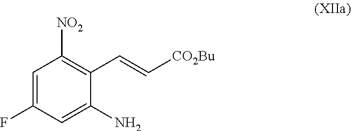

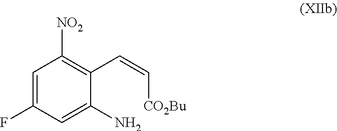

12. A compound of the following formula (XIII)

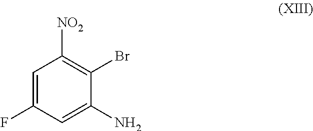 [SMILES: Nc1cc(F)cc([N+](=O)[O-])c1Br]

13. A compound of the following formula (XIV) 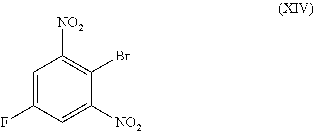 [SMILES: O=[N+]([O-])c1cc(F)cc([N+](=O)[O-])c1Br]
